(19) United States
(12) Patent Application Publication
(10) Pub. No.: US 2026/0220729 A1
(43) Pub. Date: Jul. 30, 2026

(54) PREDICTION APPARATUS, PREDICTION METHOD, AND PREDICTION PROGRAM

(71) Applicant: NIPPON TELEGRAPH AND TELEPHONE CORPORATION, Tokyo (JP)

(72) Inventor: Nobuyoshi NIHONGI, Musashino-shi, Tokyo (JP)

(73) Assignee: NIPPON TELEGRAPH AND TELEPHONE CORPORATION, Tokyo (JP)

(21) Appl. No.: 19/146,948

(22) PCT Filed: Feb. 1, 2024
(86) PCT No.: PCT/JP2024/003263

§ 371 Date: Jul. 10, 2025

(30) Foreign Application Priority Data
Feb. 2, 2023 (WO) PCT/JP2023/003474

(51) Int. Cl. G06Q 50/26

(52) U.S. Cl. CPC G06Q 50/26

(57) ABSTRACT

A prediction device includes a memory and processing circuitry configured to receive information regarding predetermined future prediction, and calculate a predicted value of a life satisfaction level by country, using the information regarding predetermined future prediction.

### TECHNICAL FIELD

The present invention relates to a prediction device, a prediction method, and a prediction program.

### BACKGROUND ART

It has become clear that climate change due to global warming affects not only changes in temperature and precipitation, but also society and economy through influences on water circulation, food production, natural disasters, and the like. To predict and evaluate these influences in a scientific manner, many efforts have been made for simulation by an integrated evaluation model integrating knowledge in various fields such as weather and human activities. In these simulations, scenarios of global warming and socio-economic activities are set, and future prediction based on models of respective fields is performed under conditions of the scenarios. Then, a comprehensive future image of the earth is predicted in such a manner that prediction results in the respective fields are matched and an economically reasonable solution is obtained (see, for example, Non Patent Literature 1 and Non Patent Literature 2).

What is predicted by the conventional integrated evaluation model is an objective indicator regarding society or economy (for example, water consumption amount, food price, food production amount, energy price, land use, or the like). Meanwhile, it is considered that it is difficult to measure human affluence only by an objective economic indicators, and in the World Happiness Report, an independently defined degree of happiness is surveyed and published on a global scale. Note that, in these surveys of the degree of happiness, subjective evaluation results by questionnaire surveys are used as important factors.

Furthermore, many studies have been conducted to evaluate what factors influence a subjective degree of happiness. For example, it is known that an average of life satisfaction levels by country, which is one item of the subjective degrees of happiness, can be predicted by a value obtained by logarithmically converting an individual's income (in other words, GDP per capita). In addition, it is known that the average of life satisfaction levels by country is influenced by an environment, a personal relationship, and the like in addition to the individual's income.

### CITATION LIST

### Non Patent Literature

Non Patent Literature 1; GCAM v5.1: representing the linkages between energy, water, land, climate, and economic systems, <https://gmd.copernicus.org/articles/12/677/2019/gmd-12-677-2019.pdf>
Non Patent Literature 2: AIM/CGE [basic] manual, <https://www.nies.go.jp/social/publications/dp/pdf/2012-01. pdf>

### SUMMARY OF INVENTION

### Technical Problem

However, there is a problem that it is difficult to utilize a subjective degree of happiness as indicators for studying measures and policies.

For example, in conventional studies, although the relationship between the present or past subjective degree of happiness (for example, the life satisfaction level by country, or the like) actually observed and other social indicators values is often examined and evaluated, studies regarding a future subjective degree of happiness have not been sufficiently performed. Therefore, in the above-described future prediction based on the integrated evaluation model, there have been cases where the results of the existing subjective degree of happiness studies are not sufficiently utilized. In addition, in the conventional degree of happiness studies, the life satisfaction level and the wellbeing index can be predicted by a logarithmic value of the individual's income (GDP per capita), but there is a problem that accuracy is not sufficient.

### Solution to Problem

Therefore, to solve the above-described problems and achieve an object, a prediction device of the present invention includes: a reception unit that receives information regarding predetermined future prediction; and a calculation unit that calculates a predicted value of a life satisfaction level by country, using the information regarding predetermined future prediction.

Further, a prediction device of the present invention includes: a reception unit that receives information regarding predetermined future prediction; and a calculation unit that calculates a predicted value of a wellbeing index, using the information regarding predetermined future prediction,

### Advantageous Effects of Invention

The present invention has an effect of enabling utilization of a subjective degree of happiness as indicators for studying measures and policies,

### BRIEF DESCRIPTION OF DRAWINGS

FIG. 1 is a diagram illustrating an outline of prediction of a life satisfaction level by country according to the present embodiment.

FIG. 2 is a diagram illustrating an example of a device configuration of a prediction device according to a first embodiment.

FIG. 3 is a table diagram illustrating an example of a coefficient of determination R2 representing accuracy of a prediction result of a predicted value of a life satisfaction level by country according to the first embodiment.

FIG. 4 is a diagram illustrating an example of a device configuration of a prediction device according to a second embodiment.

FIG. 5 is a table diagram illustrating an example of a coefficient of determination R2 representing accuracy of a prediction result of a predicted value of a life satisfaction level by country and a coefficient of determination R2 adjusted in the degree of freedom according to the second embodiment.

FIG. 6 is a diagram illustrating an example of a device configuration of a prediction device according to a third embodiment.

FIG. 7 is a table diagram illustrating an example of explanatory variables of a multiple regression model.

FIG. 8 is a diagram illustrating an example of a prediction graph of a life satisfaction level by country according to the third embodiment.

FIG. 9 is a diagram illustrating an outline of socio-economic scenarios according to the present embodiment.

FIG. 10 is a diagram for describing an outline of prediction of a life satisfaction level by country according to a fifth embodiment,

FIG. 11 is a diagram for describing an outline of prediction of a predicted value of a wellbeing index according to a sixth embodiment.

FIG. 12 is a diagram illustrating an example of a device configuration of a prediction device according to the sixth embodiment.

FIG. 13 is a table diagram illustrating an example of prediction data used for calculating the predicted value of the wellbeing index according to the sixth embodiment.

FIG. 14 is a table diagram illustrating an example of the predicted value of the wellbeing index according to the sixth embodiment.

FIG. 15 is a diagram for describing an outline of prediction of a predicted value of a wellbeing index according to a seventh embodiment.

FIG. 16 is a table diagram illustrating an example of prediction data used for calculating the predicted value of the wellbeing index according to the seventh embodiment.

FIG. 17 is a table diagram illustrating an example of a formula of a multiple regression model used for predicting the predicted value of the wellbeing index and a coefficient of determination R2 representing accuracy of a prediction result according to the seventh embodiment.

FIG. 18 is a diagram illustrating an example of a flowchart of a prediction method according to the first embodiment,

FIG. 19 is a diagram illustrating an example of a flowchart of a prediction method according to the second embodiment.

FIG. 20 is a diagram illustrating an example of a flowchart of a prediction method according to the third embodiment.

FIG. 21 is a diagram illustrating an example of a flowchart of a prediction method according to the fifth embodiment.

FIG. 22 is a diagram illustrating an example of a flowchart of a prediction method based on logarithmic conversion according to the sixth embodiment.

FIG. 23 is a diagram illustrating an example of a flowchart of a prediction method based on the multiple regression model according to the seventh embodiment.

FIG. 24 is a diagram illustrating an example of a computer on which the prediction device according to the present embodiment is implemented.

### DESCRIPTION OF EMBODIMENTS

Hereinafter, modes for carrying out the present invention (hereinafter, “embodiments”) will be described with reference to the drawings. Note that each of the embodiments is not limited to the content described below.

### [1. Outline]

A prediction device 100 of the present embodiment calculates a predicted value of a life satisfaction level for each socio-economic scenario (for example, a predicted value of a life satisfaction level by country, or the like) on the basis of a future predicted value of indicators (for example, GDP, black carbon emission amount, methane emission amount, food price, climate data, or the like) for each socio-economic scenario. As a result, the prediction device 100 enables future prediction of a subjective life satisfaction level (degree of happiness) based on an objective indicator that can be predicted for each socio-economic scenario, and enables utilization of the subjective life satisfaction level as indicators in consideration of formulation of measures and policies.

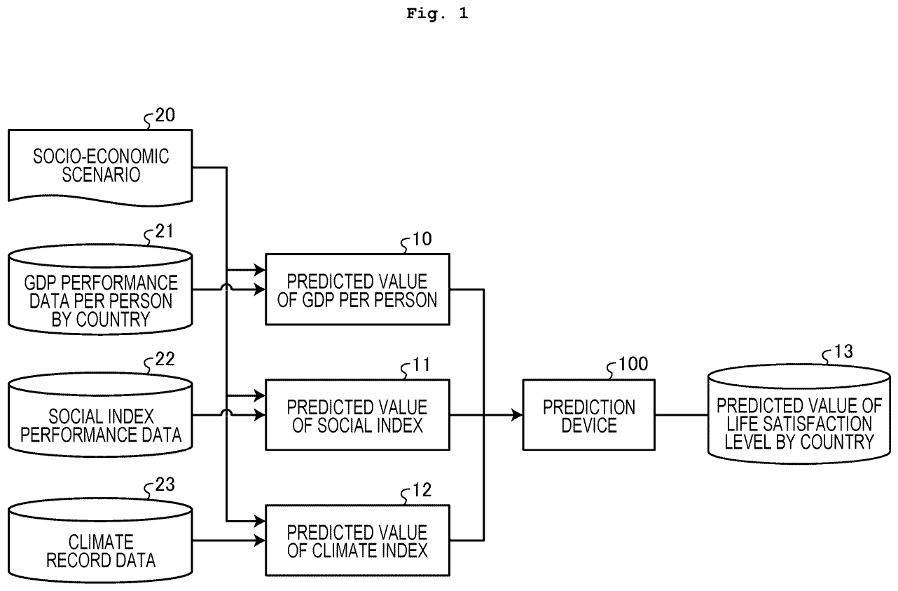

Here, a method of predicting the predicted value of a life satisfaction level by country by the prediction device 100 will be described with reference to FIG. 1. FIG. 1 is a diagram illustrating an outline of future prediction of the life satisfaction level by country according to the present embodiment. As illustrated in FIG. 1, the prediction device 100 predicts a predicted value 13 of the life satisfaction level by country using a predicted value 10 of GDP per capita, a predicted value 11 of a social indicators, and a predicted value 12 of a climate indicators. For example, the prediction device 100 can output a future predicted value of the life satisfaction level by country corresponding to a socio-economic scenario using a predicted value of an objective indicator by country (for example, GDP per capita data, Social indicators data, climate data, or the like by country) for each socio-economic scenario 20 as an input.

The above-described predicted value 10 of GDP per capita is calculated on the basis of a GDP future prediction model or the like, using GDP per capita performance data 21 by country for each socio-economic scenario 20. Note that an example of the socio-economic scenario in the present section includes shared socio-economic pathway (SSP). A plurality of SSPs is prepared assuming social and economic situations in the future, and five scenarios of SSP1, SSP2, SSP3, SSP4, and SSP5 are used. Note that the SSPs will be described in detail in a later section.

The above-described predicted value 11 of the social indicators is calculated on the basis of an integrated evaluation model GCAM or the like, using a social indicators performance data 22 for each socio-economic scenario 20, Note that the social indicators performance data referred to here includes, for example, a black carbon emission amount, a methane emission amount, a water consumption amount, a food price, a food production amount, an energy price, and a land use,

The above-described predicted value 12 of the climate indicators is calculated on the basis of a weather prediction device such as IPCC Interactive Atlas, using climate record data 23 for each socio-economic scenario 20. Note that the climate record data referred to here includes information of PM2.5 concentration and the like.

### 2. First Embodiment

Hereinafter, a first embodiment implemented by a prediction device 100 of the present embodiment will be described. The first embodiment is an embodiment of predicting a predicted value of a life satisfaction level by country using a predicted value of GDP per capita by country (hereinafter simply referred to as a “predicted value of GDP per capita”). In the first embodiment, the prediction device 100 enables prediction of the predicted value of a life satisfaction level by country with higher accuracy than the existing technique by converting the predicted value of GDP per capita on the basis of a predetermined growth curve.

### [2-1. Configuration of Prediction Device]

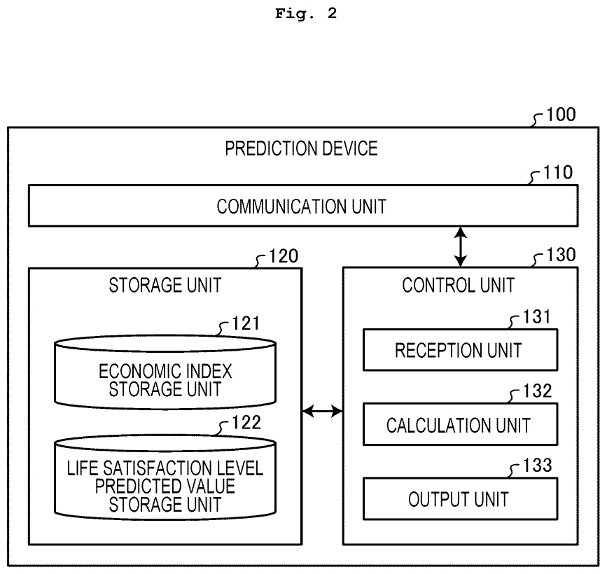

Hereinafter, a configuration example of the prediction device 100 according to the present embodiment will be described with reference to FIG. 2. FIG. 2 is a diagram illustrating an example of a device configuration of the prediction device 100 according to the first embodiment. As illustrated in FIG. 2, the prediction device 100 includes a communication unit 110, a storage unit 120, and a control unit 130.

### (Communication Unit 110)

The communication unit 110 is implemented by a network interface card (NIC) or the like, and controls communication via an electric communication line such as a local area network (LAN) or the Internet. Then, the communication unit 110 is connected to a network in a wired or wireless manner as necessary, and can bidirectionally transmit and receive information.

### (Storage Unit 120)

The storage unit 120 stores data and programs used for various types of processing by the control unit 130. Then, the storage unit 120 is implemented by a semiconductor memory element such as a random access memory (RAM) or a flash memory, or a storage device such as a hard disk or an optical disk. Further, as illustrated in FIG. 2, the storage unit 120 includes economic indicators storage unit 121 and a life satisfaction level predicted value storage unit 122.

### (Economic Index Storage Unit 121)

The economic index storage unit 121 stores information regarding the predicted value of GDP per capita used when a calculation unit 132 to be described below calculates the predicted value of a life satisfaction level by country. Note that information regarding economic indicators stored by the economic index storage unit 121 is not particularly limited as long as the information falls within the category of the economic indicators.

### (Life Satisfaction Level Predicted Value Storage Unit 122)

The life satisfaction level predicted value storage unit 122 stores the predicted value of a life satisfaction level by country calculated by a calculation unit 132 to be described below. Note that the life satisfaction level predicted value storage unit 122 can store the calculated predicted value of the life satisfaction level by country in any form. For example, the life satisfaction level predicted value storage unit 122 may store the predicted value of a life satisfaction level by country in a form such as a numerical value, a text, a graph, a formula, or a diagram without limitation.

### (Control Unit 130)

The control unit 130 includes an internal memory for temporarily storing programs and processing data defining various processing procedures and the like, and is implemented by an electronic circuit such as a central processing unit (CPU) or a micro processing unit (MPU), or an integrated circuit such as an application specific integrated circuit (ASIC) or a field programmable gate array (FPGA). Furthermore, as illustrated in FIG. 2, the control unit 130 includes a reception unit 131, a calculation unit 132, and an output unit 133.

### (Reception Unit 131)

The reception unit 131 receives information regarding predetermined future prediction. Specifically, the reception unit 131 receives the predicted value of GDP per capita as the information regarding predetermined future prediction. For example, the reception unit 131 may receive a predicted value of GDP per capita by target country in a predetermined period (for example, a predicted value of GDP per capita of Japan in 2020) as the predicted value of GDP per capita.

### (Calculation Unit 132)

The calculation unit 132 calculates the predicted value of a life satisfaction level by country, using the information regarding predetermined future prediction. Specifically, the calculation unit 132 calculates the predicted value of a life satisfaction level by country, using a growth curve based on a logistic curve or a Gompertz curve, using the predicted value of GDP per capita.

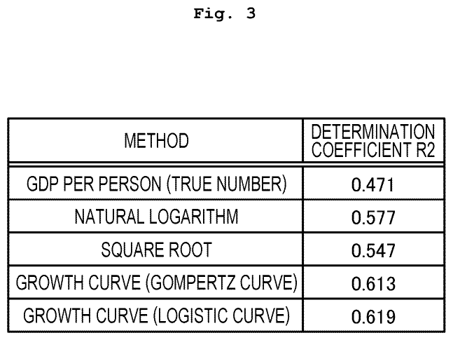

Here, creation of a prediction model of the predicted value of a life satisfaction level by country using the predicted value of GDP per capita will be described with reference to FIG. 3. FIG. 3 is a table diagram illustrating an example of a coefficient of determination R2 representing accuracy of a prediction result of the predicted value of a life satisfaction level by country according to the first embodiment.

First, as a premise for performing the prediction of FIG. 3, the predicted value of GDP per capita based on 2020 is used. Incidentally, it is conventionally known that there is a significant correlation between a logarithmic value of the GDP per capita and the life satisfaction level. In addition, it is known that growth of the life satisfaction level slows down with respect to an increase in the value of GDP per capita itself (true number before taking logarithm). In addition, it is known that the amount of change in the life satisfaction level when the GDP per capita increases by certain times is the same between a high GDP per capita and a low GDP per capita.

Note that, in the prediction by the prediction model in the first embodiment, the predicted value of a life satisfaction level by country was predicted using the predicted value of GDP per capita on the basis of a natural logarithm, a square root, a growth curve (Gompertz curve), and a growth curve (logistic curve).

As illustrated in FIG. 3, as a result of the prediction by each method, the GDP per capita (true value) had the coefficient of determination R2 of “0.471”. Further, the natural logarithm had the coefficient of determination R2 of “0.577”. Further, the square root had the coefficient of determination R2 of “0.547”, Further, the growth curve (Gompertz curve) had the coefficient of determination R2 of “0.613”. Further, the growth curve (logistic curve) had the coefficient of determination R2 of “0.619”.

As described above, in the regression based on the growth curve (logistic curve) or the growth curve (Gompertz curve) in the present embodiment, a particularly higher correlation (that is, the value of the coefficient of determination R2 was high) was observed than that in the other methods.

Hereinafter, calculation of the predicted value of a life satisfaction level by country using the growth curve based on the logistic curve will be described. The calculation unit 132 according to the first embodiment can perform calculation using Formula (1) below obtained by improving the logistic curve (for example, by adding a parameter b that enables the curve to move in an x-axis direction).

$$[ Math . 1 ]$$

$$f ( x ) = \frac{K}{1 + {e}^{- a ( x - b )}} \qquad ( 1 )$$

Note that, in Formula (1), K is a “value at which the life satisfaction level asymptotically approaches”, a is a “value representing strength of growth”, and b is a variable of the “value that enables movement in x-axis direction and simultaneously represents a position of an inflection point”. Note that, in Formula (1), when x is the predicted value of GDP per capita in an arbitrary country, and f (x) is the predicted value of the life satisfaction level.

The parameters K, a, and b in Formula (1) can be determined by curve fitting using a Levenberg-Marquardt method or the like. For example, when curve fitting based on the Levenberg-Marquardt method is performed using the performance data of the GDP per capita and the life satisfaction level in 2020, K can be defined as “7, 75054”, a can be defined as “3.97796e-05”, and b can be defined as “−5910.47939”.

With respect to the predicted value of a life satisfaction level by country calculated on the basis of Formula (1) above, for example, the coefficient of determination R2 of the predicted value of a life satisfaction level by country in 2020 is “0.577” in the existing technique (natural logarithm) as described above in FIGS. 3, and “0.619” in the calculation unit 132 according to the present embodiment. Therefore, it can be said that the calculation unit 132 can calculate the predicted value of a life satisfaction level by country with higher accuracy than in the existing technique.

Furthermore, the calculation unit 132 can use not only the logistic curve but also the growth curve based on the Gompertz curve. The calculation unit 132 according to the first embodiment can calculate the predicted value of a life satisfaction level by country with g(x), where x is the predicted value of GDP per capita in an arbitrary country, by using Formula (2) below defined using the Gompertz curve.

$$[ Math . 2 ]$$

$$g ( x ) = {Ka}^{{e}^{bx}} \qquad ( 2 )$$

Note that the parameters K, a, and b in Formula (2) can be determined by curve fitting using a Levenberg-Marquardt method or the like. For example, when curve fitting based on the Levenberg-Marquardt method is performed using the performance data of the GDP per capita and the life satisfaction level in 2020, K can be defined as “7.80590”, a can be defined as “0.542788”, and b can be defined as “−3.40392e-05”.

With respect to the predicted value of a life satisfaction level by country calculated on the basis of Formula (2) above, for example, the coefficient of determination R2 of the predicted value of a life satisfaction level by country in 2020 is “0.577” in the existing technique (natural logarithm) as described above in FIGS. 3, and “0.613” in the calculation unit 132 according to the present embodiment. Therefore, it can be said that the calculation unit 132 according to the first embodiment can calculate the predicted value of a life satisfaction level by country with higher accuracy than in the existing technique.

### (Output Unit 133)

Here, returning to FIG. 2, the description will be continued. The output unit 133 can output the predicted value of a life satisfaction level by country calculated by the calculation unit 132 in a predetermined format. For example, the output unit 133 can output the calculated predicted value of the life satisfaction level by country to a terminal device or the like operated by the user or the like in a format perceivable by the user with five senses such as a numerical value, a text, a graph, a formula, or a figure.

### 3. Second Embodiment

Hereinafter, a second embodiment implemented by a prediction device 100 of the present embodiment will be described. The second embodiment is an embodiment of predicting a predicted value of a life satisfaction level by country using a predicted value of GDP per capita and a social indicators for which future prediction is possible. For example, in the second embodiment, the prediction device 100 enables a predicted value of a life satisfaction level by country with high accuracy by constructing a multiple regression model including a social indicators (for example, a predicted value of a black carbon emission amount) for which future prediction is possible, in addition to a prediction device of GDP per capita.

### [3-1. Configuration of Prediction Device]

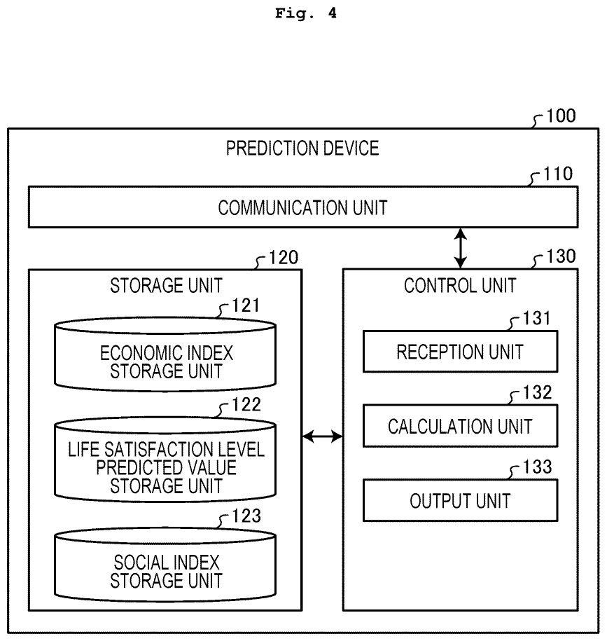

Hereinafter, a configuration example of a prediction device 100 according to the second embodiment will be described with reference to FIG. 4. FIG. 4 is a diagram illustrating an example of a device configuration of the prediction device 100 according to the second embodiment. As illustrated in FIG. 4, the prediction device 100 includes a communication unit 110, a storage unit 120, and a control unit 130.

Note that the prediction device 100 according to the second embodiment can be implemented by similar configurations and functions to those of the prediction device 100 according to the first embodiment. Therefore, in the present section, only functional units having differences between the first embodiment and the second embodiment will be described, and other descriptions will be omitted.

### (Storage Unit 120)

The storage unit 120 stores data and programs used for various types of processing by the control unit 130. In addition, the storage unit 120 is implemented by, for example, a semiconductor memory element such as a RAM or a flash memory, or a storage device such as a hard disk or an optical disc. Further, as illustrated in FIG. 4, the storage unit 120 includes an economic index storage unit 121, a life satisfaction level predicted value storage unit 122, and a social indicators storage unit 123.

(Social indicators Storage Unit 123)

The social indicators storage unit 123 stores information regarding a predicted value of a black carbon emission amount per unit area (hereinafter, simply described as “a predicted value of a black carbon emission amount”) as a social indicators used when the calculation unit 132 to be described below calculates the predicted value of a life satisfaction level by country. Note that the information regarding the social indicators stored by the social indicators storage unit 123 is not particularly limited as long as the information falls within the category of the social indicators.

### (Reception Unit 131)

The reception unit 131 receives information regarding predetermined future prediction. Specifically, the reception unit 131 receives the predicted value of GDP per capita and the predicted value of a black carbon emission amount, as the information regarding predetermined future prediction. For example, the reception unit 131 may receive a predicted value of GDP per capita by target country in a predetermined period (for example, a predicted value of GDP per capita of Japan in 2020) as the predicted value of GDP per capita. Further, the reception unit 131 may receive a predicted value of a black carbon emission amount in a predetermined period (for example, a predicted value of a black carbon emission amount in 2020, or the like) as the predicted value of a black carbon emission amount,

### (Calculation Unit 132)

The calculation unit 132 calculates the predicted value of a life satisfaction level by country, using the information regarding predetermined future prediction. Specifically, the calculation unit 132 calculates the predicted value of a life satisfaction level by country on the basis of a predetermined multiple regression model obtained by adding the predicted value of GDP per capita and the predicted value of a black carbon emission amount.

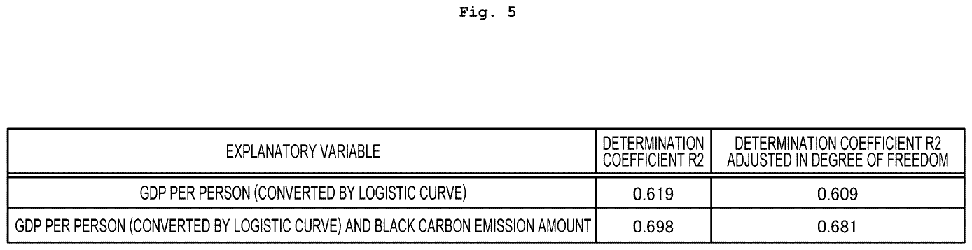

Here, creation of the prediction model of the predicted value of a life satisfaction level by country based on the predicted value of GDP per capita and the predicted value of a black carbon emission amount will be described with reference to FIG. 5. FIG. 5 is a table diagram illustrating an example of a coefficient of determination R2 representing accuracy of a prediction result of the predicted value of a life satisfaction level by country and a coefficient of determination R2 adjusted in the degree of freedom according to the second embodiment.

As a premise for performing the prediction of FIG. 5, the predicted value of a black carbon emission amount was used on the basis of a fact that a single regression analysis is performed in advance and the “predicted value of a black carbon emission amount” shows a higher correlation than other indexes (that is, the coefficient of determination R2 indicates a high value). Therefore, in the prediction by the prediction model illustrated in FIG. 5, the predicted value of a life satisfaction level by Country was predicted using the predicted value of GDP per capita (converted by a logistic curve), and the predicted value of GDP per capita (converted by a logistic curve) and the predicted value of a black carbon emission amount.

As illustrated in FIG. 5, as a result of the prediction with each explanatory variable, the predicted value of GDP per capita (converted by a logistic curve) had the coefficient of determination R2 of “0.619” and the adjusted coefficient of determination R2 of “0.609”. In addition, the predicted value of GDP per capita (converted by a logistic curve) and the predicted value of a black carbon emission amount had the coefficient of determination R2 of “0.698” and the adjusted coefficient of determination R2 of “0.681”.

As described above, in the regression based on the predicted value of GDP per capita (converted by a logistic curve) and the predicted value of a black carbon emission amount in the present embodiment, a high correlation (that is, the value of the coefficient of determination R2 is high) was observed.

Hereinafter, an example of a multiple regression model based on the predicted value of GDP per capita and the predicted value of a black carbon emission amount will be described. Formula (3) below is a formula for predicting a predicted value S of a life satisfaction level by country in consideration of an influence of the predicted value of a black carbon emission amount on the basis of Formula (1).

$$[ Math . 3 ]$$

$$S = 0.953 f ( Y ) - 5020 BC + 0.529 \qquad ( 3 )$$

Note that, in Formula (3), S is the “predicted value of a life satisfaction level by country”, Y is the “predicted value of GDP per capita”, and BC is a variable of the “predicted value of a black carbon emission amount per unit area”. In addition, a constant “0.529” in Formula (3) is calculated on the basis of a least squares method, using performance data of the GDP per capita and the life satisfaction level by country in 2020. Note that the method of calculating the constant is not limited to the least squares method.

For the predicted value S of the life satisfaction level by country calculated on the basis of Formula (3) above, for example, the coefficient of determination R2 adjusted in the degree of freedom, of the predicted value of a life satisfaction level by country in 2020 is “0.609” with the predicted value of GDP per capita (converted by a logistic curve) and is “0.681” with the predicted value of GDP and the predicted value of a black carbon emission amount, as described with reference to FIG. 5 above. Therefore, it can be said that the calculation unit 132 according to the second embodiment can calculate the predicted value of a life satisfaction level by country with higher accuracy than in the first embodiment.

### 4. Third Embodiment

Hereinafter, a third embodiment implemented by a prediction device 100 of the present embodiment will be described. The third embodiment is an embodiment of predicting a predicted value of a life satisfaction level by country using a predicted value of GDP per capita, and a social indicators for which future prediction is possible and climate data, as in the second embodiment. Note that, in the third embodiment, a prediction device 100 enables a predicted value of a life satisfaction level by country with higher accuracy by constructing a multiple regression model including a predicted value of another social indicators and a predicted value of climate data, in addition to a predicted value of GDP per capita and a predicted value of a black carbon emission amount.

### [4-1. Configuration of Prediction Device]

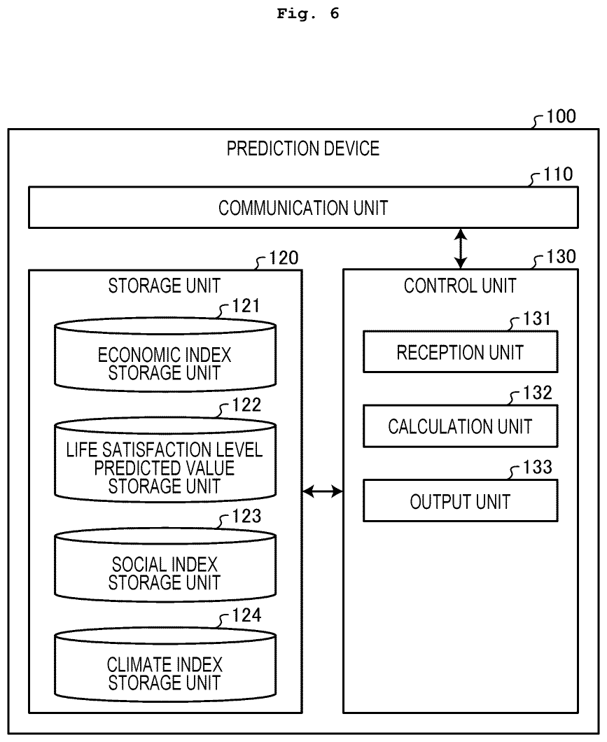

Next, a configuration example of the prediction device 100 according to the third embodiment will be described with reference to FIG. 6. FIG. 6 is a diagram illustrating an example of a device configuration of the prediction device 100 according to the third embodiment. As illustrated in FIG. 6, the prediction device 100 includes a communication unit 110, a storage unit 120, and a control unit 130.

The prediction device 100 according to the third embodiment can be implemented by similar configurations and functions to those of the prediction device 100 according to the second embodiment. Therefore, in the present section, only functional units having differences between the second embodiment and the third embodiment will be described, and other descriptions will be omitted.

### (Storage Unit 120)

The storage unit 120 stores data and programs used for various types of processing by the control unit 130. In addition, the storage unit 120 is implemented by, for example, a semiconductor memory element such as a RAM or a flash memory, or a storage device such as a hard disk or an optical disc. Further, as illustrated in FIG. 6, the storage unit 120 includes an economic index storage unit 121, a life satisfaction level predicted value storage unit 122, a social indicators storage unit 123, and a climate index storage unit 124.

(Social indicators Storage Unit 123)

The social indicators storage unit 123 stores, as a social indicators used when a calculation unit 132 to be described below calculates the predicted value of a life satisfaction level by country, information regarding a predicted value of a black carbon emission amount, information regarding a predicted value of a methane emission amount per unit area (hereinafter, simply referred to as a “predicted value of a methane emission amount”), and information regarding a predicted value of a food price. Note that information regarding the social indicators stored by the social indicators storage unit 123 is not particularly limited as long as the information falls within the category of the social indicators.

### (Climate Index Storage Unit 124)

The climate index storage unit 124 stores information regarding a predicted value of PM2.5 concentration as the climate indicators used when the calculation unit 132 to be described below calculates future prediction of a life satisfaction level by country. Note that information regarding the climate indicators stored by the climate index storage unit 124 is not particularly limited as long as the information falls within the category of the climate indicators.

### (Reception Unit 131)

A reception unit 131 receives information regarding predetermined future prediction. Specifically, the reception unit 131 receives the predicted value of GDP per capita, the predicted value of a black carbon emission amount, the predicted value of a food price, the predicted value of a methane emission amount, and the predicted value of PM2.5 concentration, as the information regarding predetermined future prediction.

For example, the reception unit 131 may receive a predicted value of GDP per capita by target country in a predetermined period (for example, a predicted value of GDP per capita of Japan in 2020) as the predicted value of GDP per capita. Further, the reception unit 131 may receive a predicted value of a black carbon emission amount in a predetermined period (for example, a predicted value of a black carbon emission amount in 2020, or the like) as the predicted value of a black carbon emission amount.

In addition, the reception unit 131 may receive a predicted value of a food price a staple food in a predetermined period (for example, a predicted value of a food price of a staple food in 2020) as the predicted value of a food price. Further, the reception unit 131 may receive a predicted value of a methane emission amount in a predetermined period (for example, a predicted value of a methane emission amount in 2020) as the predicted value of a methane emission amount. Further, the reception unit 131 may receive a predicted value of PM2.5 concentration in a predetermined period (for example, a predicted value of PM2, 5 concentration in 2020) as the predicted value of PM2.5 concentration.

### (Calculation Unit 132)

The calculation unit 132 calculates the predicted value of a life satisfaction level by country, using the information regarding predetermined future prediction. Specifically, the calculation unit 132 calculates the predicted value of a life satisfaction level by country on the basis of a predetermined multiple regression model including the predicted value of GDP per capita, and one of or a combination of a plurality of the predicted value of a black carbon emission amount, the predicted value of a food price, the predicted value of a methane emission amount, and the predicted value of PM2.5 concentration.

Note that the calculation unit 132 according to the third embodiment can calculate the predicted value of a life satisfaction level by country on the basis of Formula (3) used by the calculation unit 132 according to the second embodiment. In that case, the calculation unit 132 can use Formula (3) by replacing a variable BC of Formula (3) from the “predicted value of a black carbon emission amount per unit area” to the “predicted value of a methane emission amount per unit area”, the “predicted value of a food price”, and the “predicted value of PM2.5 concentration”.

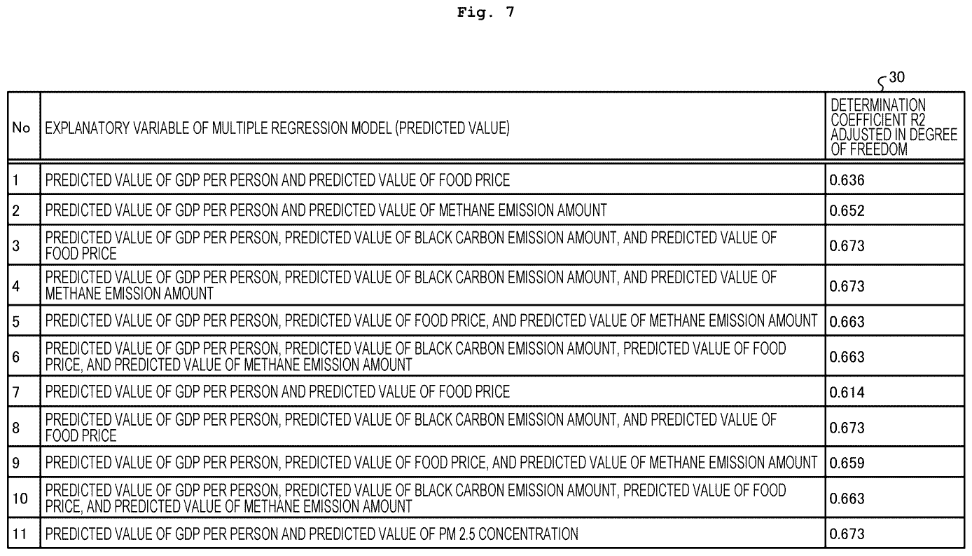

Here, a coefficient of determination R2 adjusted in the degree of freedom according to the third embodiment will be described with reference to FIG. 7. FIG. 7 is a table diagram illustrating an example of the explanatory variables of the multiple regression model. A list 30 illustrated in FIG. 7 includes “No”, “explanatory variables (predicted values) of the multiple regression model”, and the “coefficient of determination R2 adjusted in the degree of freedom” as items.

Note that, as content included in the item of the explanatory variables of the multiple regression model, the predicted value of GDP per capita means a “predicted value of GDP per capita converted by a growth curve”. Further, the predicted value of a food price means a “predicted value of a food price of a staple food”. Further, the predicted value of a black carbon emission amount means a “predicted value of a black carbon emission amount per unit area”. Further, the predicted value of a methane emission amount means a “predicted value of a methane emission amount per unit area”.

For example, in the explanatory variables of the “predicted value of GDP per capita and predicted value of a food price” of the multiple regression model identified by No of “1”, the coefficient of determination R2 adjusted in the degree of freedom is “0.636”. This numerical value is higher than “0.609” in the above-described existing technique (natural logarithm). In addition, with respect to the other items as well, the coefficient of determination R2 adjusted in the degree of freedom is higher in value than “0.577” in the above-described existing technique (natural logarithm), it can be said that the calculation unit 132 according to the third embodiment can calculate the predicted value of a life satisfaction level by country with higher accuracy than the existing technique.

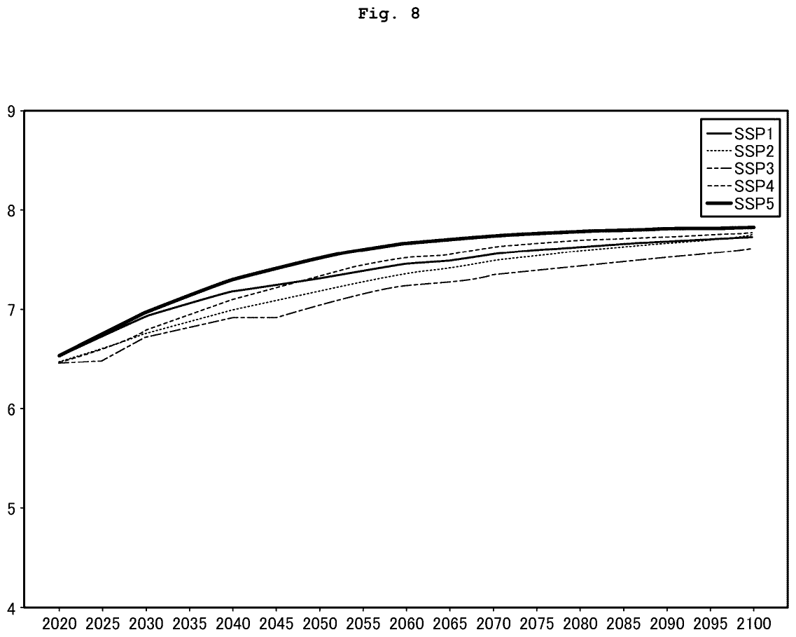

Next, a prediction result of the life satisfaction level by country by the prediction device 100 according to the third embodiment will be described with reference to FIG. 8. FIG. 8 is a diagram illustrating an example of a prediction graph of the life satisfaction level by country according to the third embodiment.

As illustrated in FIG. 8, the prediction device 100 according to the third embodiment can predict a life satisfaction level in Japan from 2020 to 2100 for each of socio-economic scenarios (SSP1 to SSP5) as the prediction of the life satisfaction levels by country. Therefore, the prediction device 100 can predict how the life satisfaction level by country in a certain future period changes for each socio-economic scenario.

The prediction device 100 can predict the predicted value of a life satisfaction level by country without limiting the prediction target country by using the predicted value of GDP per capita of the target country for the input information. Note that, in FIG. 8, the prediction device 100 predicts the predicted value of a life satisfaction level by country in the period from 2020 to 2100, but the predictable period is not limited thereto.

### 5. Fourth Embodiment

Hereinafter, a fourth embodiment implemented by a prediction device 100 of the present embodiment will be described. The fourth embodiment is an embodiment of predicting a predicted value of a life satisfaction level by country on the basis of a predicted value for each socio-economic scenario.

As the predicted value of GDP per capita, the predicted value of a black carbon emission amount, the predicted value of a methane emission amount, the predicted value of a food price, the predicted value of PM2.5 concentration, and the like, which are input values to the prediction device 100 described above, predicted values for each socio-economic scenario are known.

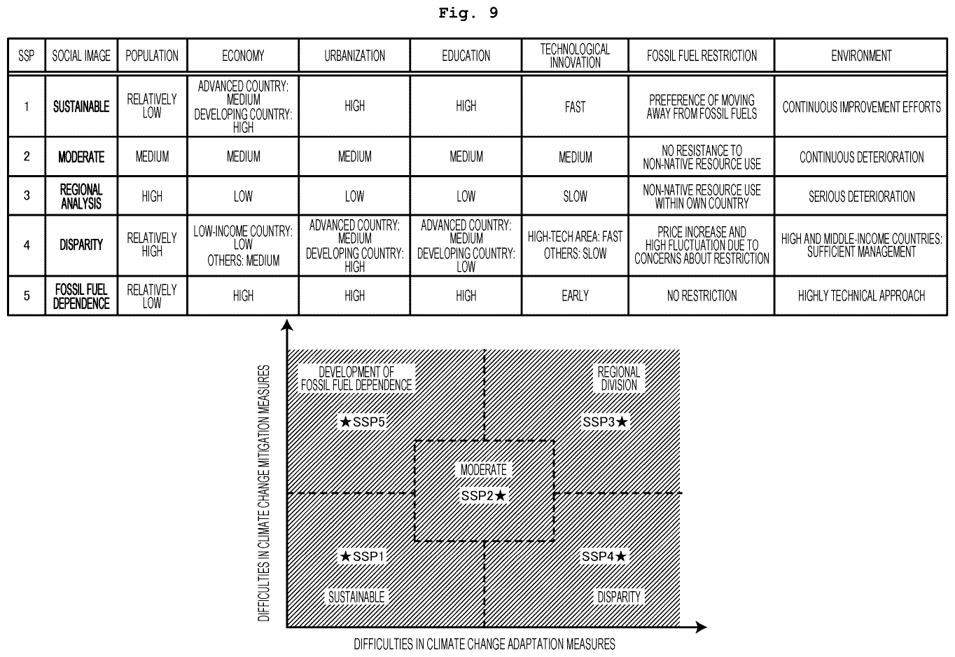

The socio-economic scenario described herein is prepared by assuming a plurality of social and economic situations in the future, and five scenarios SSP1 to SSP5 are used, Here, an outline of the socio-economic scenarios will be described with reference to FIG. 9. FIG. 9 is a diagram illustrating an outline of the socio-economic scenarios according to the present embodiment.

As illustrated in the upper diagram of FIG. 9, SSP is divided into five social images of SSP1 as “sustainability”, SSP2 as “middle of the road”, SSP3 as “regional rivalry”, SSP4 as “inequality”, and SSP5 as “fossil-fueled development”. Items for each social image include “population”, “economy”, “urbanization”, “education”, “technological innovation”, “fossil fuel restriction”, and “environment”. For example, when SSP is “1”, the social image is “sustainability”, the population is “relatively low”, the economy is “advanced country: medium, developing country; high”, the urbanization is “high”, the education is “high”, the technological innovation is “fast”, the fossil fuel restriction is “preference of moving away from fossil fuels”, and the environment is “continuous improvement efforts”. Furthermore, these five SSPs are plotted in a matrix in which the vertical axis is “difficulties in climate change mitigation measures” and the horizontal axis is “difficulties in climate change adaptation measures” as illustrated in the lower diagram of FIG. 9.

The prediction device 100 of the present embodiment can also be applied to socio-economic scenarios with different policies and measures as long as they are data from which the future prediction of GDP per capita and a social indicators or climate data can be obtained besides the above-described SSP1 to SSP5.

For example, a regression coefficient of the predicted value of the black carbon emission amount is a negative value in the above-described multiple regression model, depending on the socio-economic scenario to be used. This means that the life satisfaction level is further improved by the society selecting an aspect of the society in which the black carbon emission amount decreases. As described above, the prediction device 100 according to the fourth embodiment is not limited to SSPs, and enables users to know how the life satisfaction level changes by using various socio-economic scenarios to select what kind of Society.

### [5-1. Configuration of Prediction Device]

The prediction device 100 according to the fourth embodiment can be implemented by similar configurations and functions to those of the prediction device 100 according to the first to third embodiments. Therefore, in the present section, only functional units having differences will be described, and other descriptions will be omitted.

(Reception unit 131)

A reception unit 131 receives information regarding predetermined future prediction. Specifically, the reception unit 131 according to the fourth embodiment receives the information regarding predetermined future prediction predicted for each of a plurality of socio-economic scenarios. For example, the reception unit 131 can receive a socio-economic scenario other than SSPs as the information regarding predetermined future prediction. Then, the prediction device 100 according to each embodiment can predict the predicted value of a life satisfaction level by country on the basis of the received socio-economic scenario other than SSPs. Note that the reception unit 131 according to the fourth embodiment can receive the socio-economic scenario without limitation as long as it falls within the category of the socio-economic scenario.

### 6. Fifth Embodiment

Hereinafter, a fifth embodiment implemented by a prediction device 100 of the present embodiment will be described. The fifth embodiment is another form of prediction of the predicted value of a life satisfaction level by country using the predicted value of GDP per capita described in the section of the first embodiment.

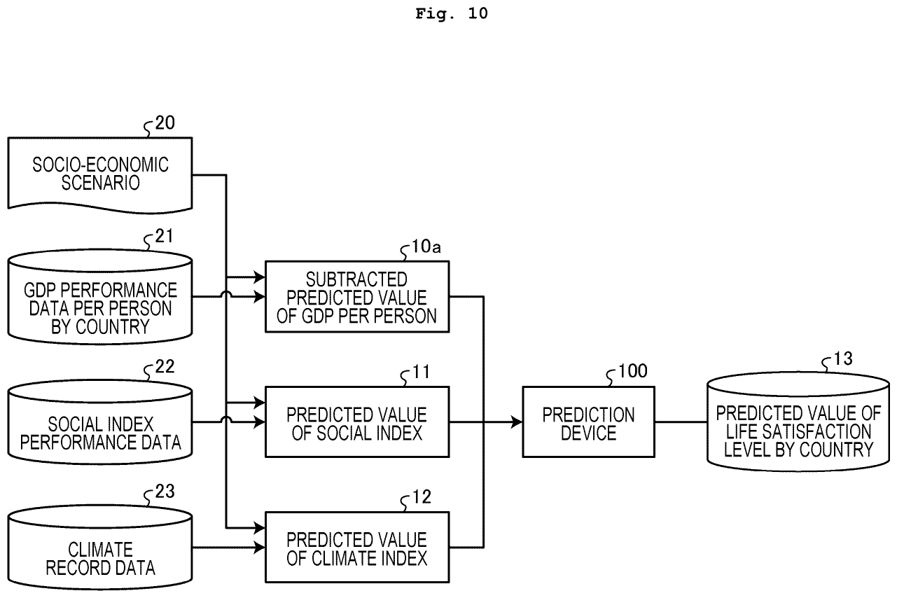

First, a method of predicting the predicted value of a life satisfaction level by country by the prediction device 100 will be described with reference to the drawings. FIG. 10 is a diagram for describing an outline of the prediction of a life satisfaction level by country according to the fifth embodiment. As illustrated in FIG. 10, the prediction device 100 according to the fifth embodiment outputs a future predicted value of a life satisfaction level by country corresponding to a socio-economic scenario by using a predicted value obtained by subtracting a “cost incurred due to climate change (hereinafter may be referred to as “climate change cost”}” from the predicted value of GDP per capita used in the first embodiment (subtracted predicted value 10a of GDP per capita) as an input.

The prediction device 100 can predict a predicted value to which an influence of climate change is added by subtracting the climate change cost as described above. As a result, the prediction device 100 can predict a predicted value with higher accuracy than the predicted value of a life satisfaction level by country predicted in the first embodiment,

### [6-1. Configuration of Prediction Device]

Next, a configuration example of the prediction device 100 according to the fifth embodiment will be described. Note that a device configuration of the prediction device 100 according to the fifth embodiment is similar to the device configuration of the prediction device 100 according to the first embodiment, and thus will be described with reference to FIG. 2 again. Further, in the present section, only functional units having differences will be described, and other descriptions will be omitted.

### (Economic Index Storage Unit 121)

An economic index storage unit 121 stores information regarding the predicted value of GDP per capita and information regarding the climate change cost.

### (Reception Unit 131)

A reception unit 131 according to the fifth embodiment receives the predicted value of GDP per capita and the climate change cost as the information regarding predetermined future prediction. For example, the reception unit 131 receives, as the climate change cost, a climate change cost calculated on the basis of a risk ratio of agricultural productivity, coastal water immersion, flood, occupational health cost, thermal power generation amount, water immersion, or the like (see, for example, Reference Literature 1).

(Reference Literature 1): Total economic costs of climate change at different discount rates for market and non-market values <URL: https://iopscience. iop. org/article/10.1088/1748-9326/accdee>, <Retrieved on Jan. 10, 2024>

### (Calculation Unit 132)

A calculation unit 132 according to the fifth embodiment subtracts the climate change cost from the predicted value of GDP per capita. Then, the calculation unit 132 calculates the predicted value of a life satisfaction level by country, using a growth curve based on a logistic curve or a Gompertz curve, using the predicted value of GDP per capita from which the climate change cost has been subtracted.

Note that the calculation unit 132 according to the fifth embodiment calculates the predicted value of a life satisfaction level by country by a similar method to the first embodiment, and thus detailed description thereof is omitted in the present section.

### 7. Sixth Embodiment

Hereinafter, a sixth embodiment implemented by a prediction device 100 of the present embodiment will be described. The sixth embodiment is an embodiment of predicting a predicted value of a wellbeing index using a predicted value of GDP per capita in addition to a predicted value of a life satisfaction level by country.

As described above, many studies have been conducted to evaluate what factors influence a subjective degree of happiness. There are various indices of the subjective degree of happiness, and an index called a “wellbeing index” is also used in addition to the life satisfaction level by country described in the first to fifth embodiments. Wellbeing means being in a physically and socially good state in which an individual's right or self-realization is guaranteed (that is, a state of being satisfied), and the wellbeing index is an index of the above-described state.

Similarly to the life satisfaction level by country, there is a problem that prediction accuracy of the wellbeing index is not sufficient. As a result, there is a problem that it is difficult to utilize the wellbeing index as an index for studying measures and policies.

Therefore, the prediction device 100 according to the sixth embodiment calculates a predicted value of the wellbeing index on the basis of logarithmic conversion of a predicted value of GDP per capita.

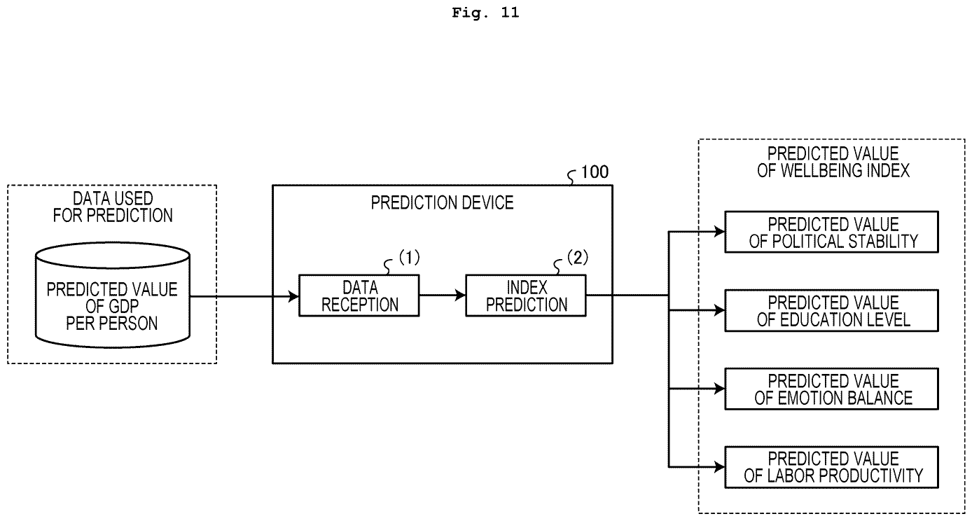

Here, an outline of processing by the prediction device 100 according to the sixth embodiment will be described with reference to the drawings. FIG. 11 is a diagram for describing an outline of prediction of the predicted value of the wellbeing index according to the sixth embodiment.

The prediction device 100 receives an input of the predicted value of GDP per capita as prediction data ((1) in FIG. 11). Next, the prediction device 100 predicts the predicted value of the wellbeing index such as a predicted value of political stability, a predicted value of an education level, a predicted value of emotion balance, and a predicted value of labor productivity on the basis of the logarithmic conversion of the received predicted value of GDP per capita ((2) in FIG. 11),

As described above, the prediction device 100 can accurately predict the predicted value of the wellbeing index by using the input predicted value of GDP per capita. Therefore, the prediction device 100 according to the sixth embodiment has an effect of enabling utilization of the wellbeing index as an index for studying measures and policies,

### [7-1. Configuration of Prediction Device]

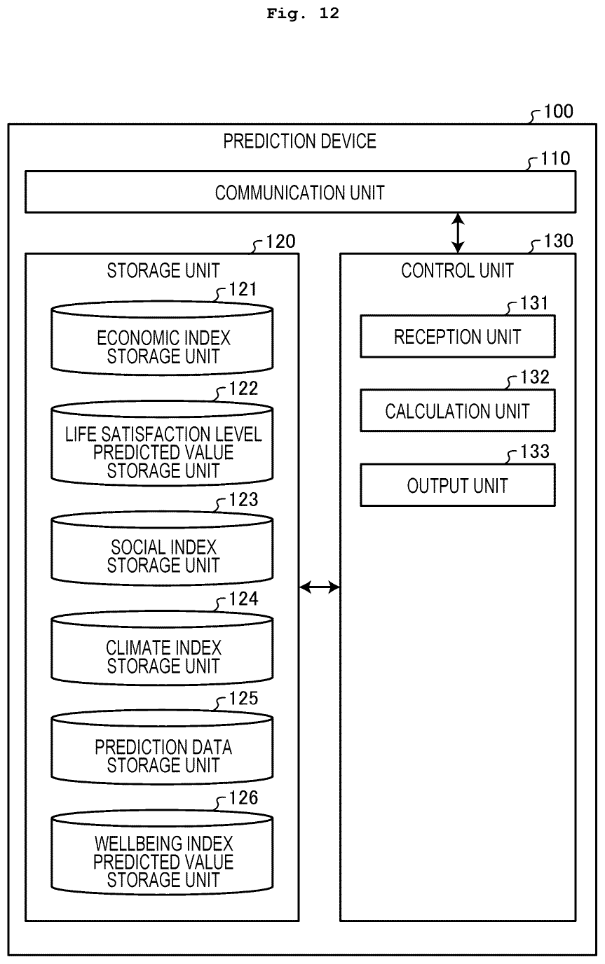

Hereinafter, a configuration example of the prediction device 100 according to the sixth embodiment will be described. FIG. 12 is a diagram illustrating an example of a device configuration of the prediction device 100 according to the sixth embodiment. As illustrated in FIG. 12, the prediction device 100 according to the sixth embodiment includes a communication unit 110, a storage unit 120, and a control unit 130. Note that the prediction device 100 according to the sixth embodiment can be implemented by similar configurations and functions to those of the prediction devices 100 according to the first to fifth embodiments. Therefore, in the present section, only functional units having differences will be described, and other descriptions will be omitted.

### (Storage Unit 120)

As illustrated in FIG. 12, the storage unit 120 according to the sixth embodiment includes an economic index storage unit 121, a life satisfaction level predicted value storage unit 122, a social indicators storage unit 123, a climate index storage unit 124, a prediction data storage unit 125, and a wellbeing index predicted value storage unit 126.

### (Prediction Data Storage Unit 125)

The prediction data storage unit 125 stores the predicted value of GDP per capita as predetermined prediction data used when a calculation unit 132 to be described below predicts the predicted value of the wellbeing index. Note that, in the first to fifth embodiments, it has been described that the predicted value of GDP per capita is stored by the economic index storage unit 121, whereas in the following sixth and seventh embodiments, it will be described that the prediction data storage unit 125 stores the predicted value of GDP per capita for convenience of description of the embodiments.

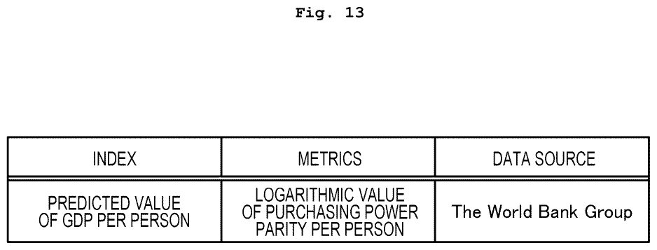

Here, the prediction data stored by the prediction data storage unit 125 will be described with reference to a table diagram. FIG. 13 is a table diagram illustrating an example of the prediction data used for calculating the predicted value of the wellbeing index according to the sixth embodiment.

FIG. 13 illustrates a metric and a data source for each prediction data stored by the prediction data storage unit 125. As illustrated in FIG. 13, the predicted value of GDP per capita is a metric of a “logarithmic value of purchasing power parity per person”, and for example, “The World Bank Group (see, for example, Reference Literature 2)” or the like is used as a data source.

(Reference Literature 2) The World Bank Group World Development Indicators, <URL: https://databank. worldbank. org/source/world-development-indicators>, <Retrieved on Jan. 10, 2024> (Wellbeing Index Predicted Value Storage Unit 126)

The wellbeing index predicted value storage unit 126 stores the predicted value of political stability, the predicted value of an education level, the predicted value of emotion balance, and the predicted value of labor productivity as the predicted values of the wellbeing indices calculated by the calculation unit 132 to be described below.

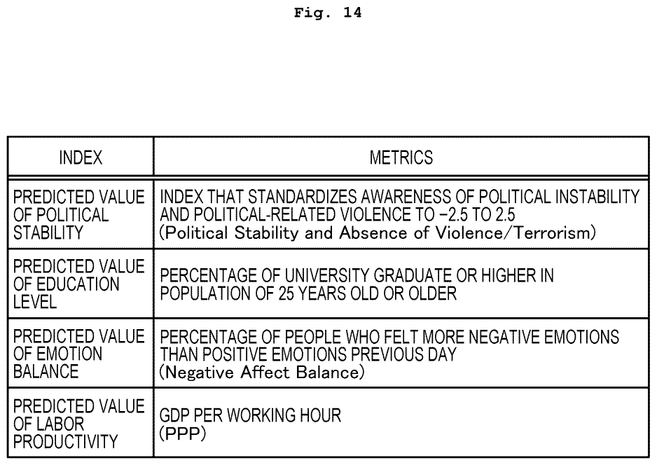

Here, the predicted value of the wellbeing index stored by the wellbeing index predicted value storage unit 126 will be described with reference to a table, FIG. 14 is a table diagram illustrating an example of the predicted value of the wellbeing index according to the sixth embodiment. FIG. 14 illustrates metrics and data sources of the respective predicted values of the wellbeing indexes stored by the wellbeing index predicted value storage unit 126.

As illustrated in FIG. 14, the predicted value of political stability is a metric of an Windex that standardizes awareness of political instability and political-related violence to −2.5 to 2.5 (political stability and absence of violence/terrorism) “. The predicted value of an education level is a metric of a “percentage of university graduate or higher in population of 25 years old or older”. The predicted value of emotion balance is a metric of a “percentage of people who felt more negative emotions than positive emotions previous day (negative affect balance)”. The predicted value of labor productivity is a metric of “GDP (PPP) per working hour”.

### (Reception Unit 131)

A reception unit 131 according to the sixth embodiment receives the predicted value of GDP per capita as information regarding predetermined future prediction (prediction data). Then, the reception unit 131 stores the received predicted value of GDP per capita in the prediction data storage unit 125.

### (Calculation Unit 132)

The calculation unit 132 according to the sixth embodiment calculates the predicted value of the wellbeing index, using information regarding predetermined future prediction (prediction data). Specifically, the calculation unit 132 calculates at least one of the predicted value of political stability, the predicted value of labor productivity, the predicted value of emotion balance, or the predicted value of an education level as the predicted value of the wellbeing index on the basis of logarithmic conversion of the predicted value of GDP per capita received by the reception unit 131. Note that the wellbeing index, which is one item of the above-described subjective degree of happiness, can be predicted with a value obtained by logarithmically converting an individual's income (in other words, GDP per capita).

### 8. Seventh Embodiment

Hereinafter, a seventh embodiment implemented by a prediction device 100 of the present embodiment will be described. The seventh embodiment is another embodiment of the sixth embodiment, and is an embodiment of predicting a predicted value of a wellbeing index on the basis of a multiple regression model obtained by combining a plurality of pieces of prediction data in addition to a predicted value of GDP per capita.

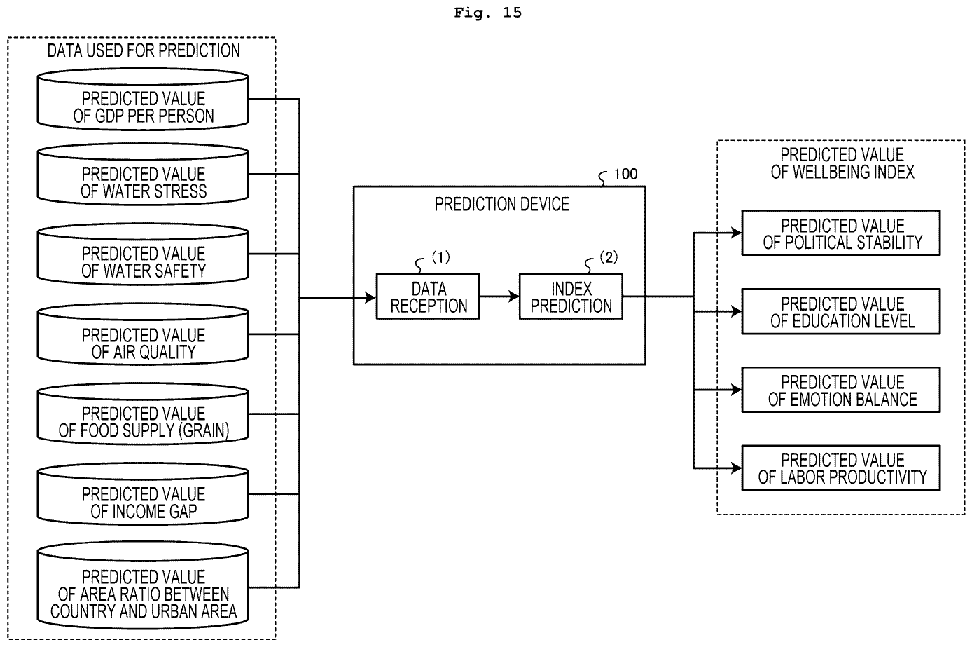

Here, an outline of processing by the prediction device 100 according to the seventh embodiment will be described with reference to the drawings. FIG. 15 is a diagram for describing an outline of prediction of a predicted value of a wellbeing index according to the seventh embodiment.

The prediction device 100 receives an input of data used for prediction such as a predicted value of water stress, a predicted value of water safety, a predicted value of air quality, a predicted value of food supply (grain), a predicted value of an income inequality, and a predicted value of an area ratio between a rural area and an urban area, in addition to the predicted value of GDP per capita ((1) in FIG. 15).

Next, the prediction device 100 constructs a multiple regression model by combining received data such as the predicted value of GDP per capita, the predicted value of water stress, the predicted value of water safety, the predicted value of air quality, the predicted value of food supply (grain), the predicted value of GDP per capita, the predicted value of an income inequality, and the predicted value of an area ratio between a rural area and an urban area, and predicts the predicted value of the wellbeing index such as a predicted value of political stability, a predicted value of education level, a predicted value of emotion balance, and a predicted value of labor productivity on the basis of the multiple regression model ((2) in FIG. 15).

As described above, the prediction device 100 can accurately predict the predicted value of the wellbeing index using the plurality of pieces of input prediction data. Therefore, the prediction device 100 according to the seventh embodiment has an effect of enabling utilization of the predicted value of the wellbeing index as an index for studying measures and policies.

### [8-1. Configuration of Prediction Device]

Hereinafter, a configuration example of the prediction device 100 according to the seventh embodiment will be described. Note that the prediction device 100 according to the seventh embodiment can be implemented by similar configuration and function to those of the prediction device 100 according to the sixth embodiment. Therefore, the configuration of the prediction device 100 according to the seventh embodiment will be described with reference to FIG. 12 again. Note that, in the present section, only functional units having differences will be described, and other descriptions will be omitted.

### (Prediction Data Storage Unit 125)

A prediction data storage unit 125 stores predetermined data used when a calculation unit 132 to be described below predicts the predicted value of the wellbeing index. Specifically, the prediction data storage unit 125 stores the predicted value of GDP per capita, the predicted value of water stress, the predicted value of water safety, the predicted value of air quality, the predicted value of food supply (grain), the predicted value of an income inequality, and the predicted value of an area ratio of a rural area and an urban area.

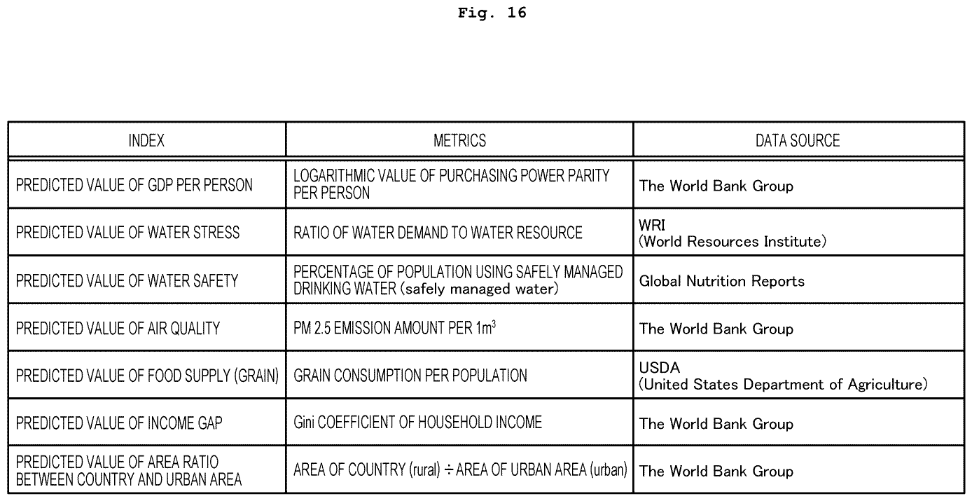

Here, the prediction data stored by the prediction data storage unit 125 will be described with reference to a table diagram. FIG. 16 is a table diagram illustrating an example of the prediction data used for calculating the predicted value of the wellbeing index according to the seventh embodiment. FIG. 16 illustrates a metric and a data source for each prediction data stored by the prediction data storage unit 125. Note that the item of the predicted value of GDP per capita has similar content to the description in FIG. 13, and thus description in the present section is omitted.

As illustrated in FIG. 16, the predicted value of water stress is a metric of a “ratio of water demand to water resource”, and for example, “World Resources Institute (WRI) (see, for example, Reference Literature 3)” or the like is used as a data source.

Further, the predicted value of water safety is a metric of a “percentage of population using safely managed drinking water (safely managed water) “, and for example, “Global Nutrition Reports (see, for example, Reference Literature 4)” or the like is used as a data source.

Further, the predicted value of air quality is a metric of a “PM2.5 emission amount per cubic meter”, and for example, “The World Bank Group (see, for example, Reference Literature 2)” or the like is used as a data source.

Furthermore, the predicted value of food supply (grain) is a metric of a “grain consumption per population”, and for example, “United States Department of Agriculture (USDA) (see, for example, Reference Literature 5)” or the like is used as a data source.

Furthermore, the predicted value of an area ratio between a rural area and an urban area is a metric of “the area of the rural area (rural)+the area of the urban area (urban) “, and for example, “The World Bank Group (see, for example, Reference Literature 2)” or the like is used as a data source.

(Reference Literature 3) WRI (World Resources Institute) AQUEDUCT COUNTRY RANKINGS, <URL: https://www.wri. org/applications/aqueduct/country-rankings/>, <Retrieved on Jan. 10, 2024>

(Reference Literature 4) Global Nutrition Reports, <URL: https://globalnutritionreport.org/reports/2022-global-nutrition-report/dataset-metadata/>, <Retrieved on Jan. 10, 2024>

(Reference Literature 5) United States Department of Agriculture Foreign Agricultural Service, <URL: https://apps.fas. usda. gov/psdonline/app/index. html #/ap p/downloads>, <Retrieved on Jan. 10, 2024>

Note that the above-described data for prediction is merely an example. For example, the prediction data storage unit 125 can store an index regarding physical health such as a questionnaire result of self-evaluation (questionnaire result of self-evaluation regarding health), an index regarding mental health such as a suicide rate, an index representing a comprehensive health state such as disability-adjusted life years (DALY), and the like, as the prediction data in addition to the above-described data.

In the present embodiment, the DALY is an index for measuring an overall disease burden in a country or region, or a disease burden regarding a specific disease in the Country or region. For example, the index is used in international comparison of DALY per 100,000 people, and is an index in which the number of years of a death earlier than an ideal life and the number of years with a disease even if not leading to the death multiplied by a coefficient (<1.0) according to the degree of the disease are calculated as a burden.

### (Reception Unit 131)

A reception unit 131 according to the seventh embodiment receives the predicted value of GDP per capita, the predicted value of water stress, the predicted value of water safety, the predicted value of air quality, the predicted value of food supply, the predicted value of an income inequality, and the predicted value of an area ratio between a rural area and an urban area as the information regarding predetermined future prediction (prediction data). Then, the reception unit 131 stores the received predicted value of GDP per capita, the received predicted value of water stress, the received predicted value of water safety, the received predicted value of air quality, the received predicted value of food supply, the received predicted value of an income inequality, and the received predicted value of an area ratio between a rural area and an urban area in the prediction data storage unit 125.

Note that the reception unit 131 can receive, in addition to the above-described prediction data, predicted values of the index regarding physical health such as a questionnaire result of self-evaluation (questionnaire result of self-evaluation regarding health), the index regarding mental health such as a suicide rate, the index representing a comprehensive health state such as DALY, and the like.

### (Calculation Unit 132)

A calculation unit 132 according to the seventh embodiment calculates the predicted value of the wellbeing index, using the information regarding predetermined future prediction (prediction data). Specifically, the calculation unit 132 calculates at least one of the predicted value of political stability, the predicted value of labor productivity, the predicted value of emotion balance, or the predicted value of an education level as the predicted value of the wellbeing index on the basis of the multiple regression model including one piece of or a combination of a plurality of pieces of the information regarding predetermined future prediction (prediction data) received by the reception unit 131. For example, the calculation unit 132 calculates the predicted value of the wellbeing index on the basis of the predetermined multiple regression model including the predicted value of GDP per capita, and one of or a combination of a plurality of the predicted value of water stress, the predicted value of water safety, the predicted value of air quality, the predicted value of food supply, the predicted value of an income inequality, and the predicted value of an area ratio between a rural area and an urban area.

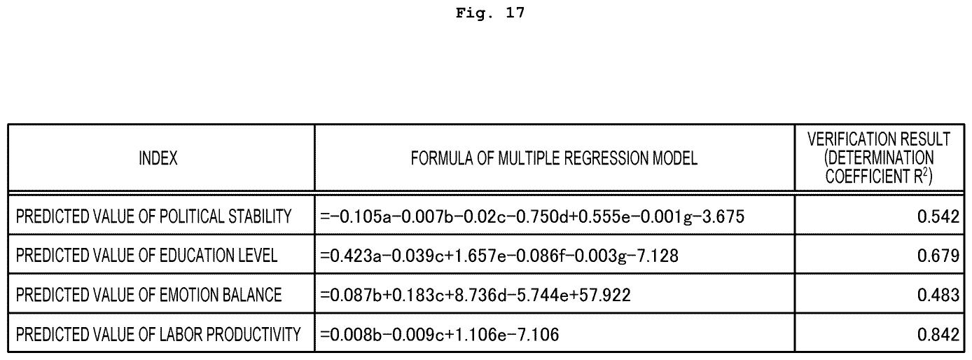

Here, calculation of the predicted value of the wellbeing index by the calculation unit 132 will be described with reference to the drawings. FIG. 17 is a table diagram illustrating an example of a formula of the multiple regression model used for predicting the predicted value of the wellbeing index and a coefficient of determination R2 representing accuracy of a prediction result according to the seventh embodiment. FIG. 17 illustrates the formula of the multiple regression model used when the calculation unit 132 calculates the predicted value of the wellbeing index in association with each wellbeing index.

The calculation unit 132 derives a multiple regression model with an actual index of a predicted value of political stability given as a data source by “The World Bank Group (see, for example, Reference Literature 2)” as an objective variable and “the predicted value of water stress (a), the predicted value of water safety (b), the predicted value of air quality (c), the predicted value of food supply (grain) (d), the predicted value of GDP per capita (e), and the predicted value of area ratio between a rural area and an urban area (g)” as explanatory variables. Then, the calculation unit 132 inputs numerical values of “the predicted value of water stress (a), the predicted value of water safety (b), the predicted value of air quality (c), the predicted value of food supply (grain) (d), the predicted value of GDP per capita (e), and the predicted value of area ratio between a rural area and an urban area (g)”, which are the explanatory variables, to the derived multiple regression model, to calculate the predicted value of political stability as the predicted value of the wellbeing index.

For example, the calculation unit 132 calculates the “predicted value of political stability” on the basis of “=−0.105a-0.007b-0.02c-0,750d+0.555e-0.001g-3.675” as the formula of the multiple regression model. Note that “−3.675” is a constant calculated in advance. Note that the coefficient of determination R2 of the formula of the multiple regression model described above is “0.542”.

Furthermore, the calculation unit 132 derives a multiple regression model with an actual index of a predicted value of an education level given as a data source by “The World Bank Group (see, for example, Reference Literature 2)” as an objective variable and “the predicted value of water stress (a), the predicted value of air quality (c), the predicted value of GDP per capita (e), the predicted value of an income inequality (f), and the predicted value of an area ratio between a rural area and an urban area (g)” as explanatory variables. Then, the calculation unit 132 inputs numerical values of “the predicted value of water stress (a), the predicted value of air quality (c), the predicted value of GDP per capita (e), the predicted value of an income inequality (f), and the predicted value of an area ratio between a rural area and an urban area (g)”, which are the explanatory variables, to the derived multiple regression model to calculate the predicted value of an education level as the predicted value of the wellbeing index.

For example, the calculation unit 132 calculates the “predicted value of an educational level” on the basis of “−0.423a−0.039c+1.657e−0.086f−0.003g−7.128” as the formula of the multiple regression model. Note that “−7.128” is a constant calculated in advance. Note that the coefficient of determination R² of the formula of the multiple regression model described above is “0.679”.

Furthermore, the calculation unit 132 derives a multiple regression model with an actual index of a predicted value of emotion balance given as a data source by “OECD How's Life? Well-Being (see, for example, Reference Literature 6)” as an objective variable and “the predicted value of water safety (b), the predicted value of air quality (c), the predicted value of food supply (grain) (d), and the predicted value of GDP per capita (e)” as explanatory variables. Then, the calculation unit 132 inputs numerical values of “the predicted value of water safety (b), the predicted value of air quality (c), the predicted value of food supply (grain) (d), and the predicted value of GDP per capita (e)”, which are the explanatory variables, to the derived multiple regression model to calculate the predicted value of emotion balance as the predicted value of the wellbeing index.

For example, the calculation unit 132 calculates the “predicted value of emotion balance” on the basis of “=0.087b+0.183c+8.736d−5.744e+57.922” as the formula of the multiple regression model. Note that “+57.922” is a constant calculated in advance. Note that the coefficient of determination R² of the formula of the multiple regression model described above is “0.483”.

(Reference Literature 6) OECD How's Life? Well-Being, <URL: https://stats. oecd. org/Index. aspx?DataSet Code=HSL>, <Retrieved on Jan. 10, 2024>

Further, the calculation unit 132 derives a multiple regression model with an actual index of a predicted value of labor productivity given as a data source by “International Labour Organization (ILO) (see, for example, Reference Literature 7)” as an objective variable and “the predicted value of water safety (b), the predicted value of air quality (c), and the predicted value of GDP per capita (e)” as explanatory variables. Then, the calculation unit 132 inputs the numerical value of “the predicted value of water safety (b), the predicted value of air quality (c), and the predicted value of GDP per capita (e) “, which are the explanatory variables, to the derived multiple regression model to calculate the predicted value of labor productivity as the predicted value of the wellbeing index.

For example, the calculation unit 132 calculates the “predicted value of labor productivity” on the basis of “=0.008b-0.009c+1.106e-7,106” as the formula of the multiple regression model. Note that “−7,106” is a constant calculated in advance. Note that the coefficient of determination R² of the formula of the multiple regression model described above is “0.842”.

(Reference Literature 7) ILOSTAT Statistics on labour productivity, <URL: https://ilostat. ilo. org/topics/labour-productivity/>, <Retrieved on Jan. 10, 2024>

Note that the calculation unit 132 can calculate the predicted value of the wellbeing index on the basis of a multiple regression model including the index regarding physical health such as a questionnaire result of self-evaluation (questionnaire result of self-evaluation regarding health), the index representing comprehensive health state such as DALY, and the index regarding mental health such as a suicide rate, in addition to the above-described multiple regression model.

### [9. Processing Procedure]

A processing procedure of the prediction device 100 will be described below, Flowcharts for the respective embodiments will be described for the “first embodiment”, “second embodiment”, “third embodiment”, “fifth embodiment”, “sixth embodiment”, and “seventh embodiment”. The fourth embodiment is similar to the first embodiment, the second embodiment, and the third embodiment, and thus will not be described. Note that the processes to be described below may be executed in a different order or processing may be omitted. In addition, the processing procedures of the embodiments may be appropriately combined and performed.

### First Embodiment

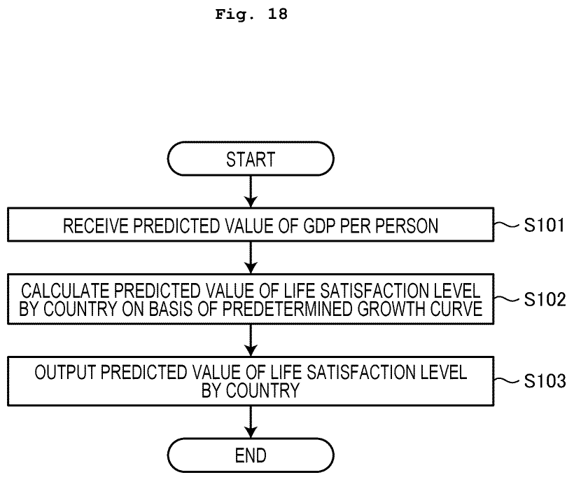

First, the processing procedure of the first embodiment will be described with reference to FIG. 18. FIG. 18 is a diagram illustrating an example of the flowchart of the prediction method according to the first embodiment.

The reception unit 131 receives the predicted value of GDP per capita (process S101). The calculation unit 132 calculates the predicted value of a life satisfaction levels by country on the basis of the predetermined growth curve (process S102). For example, the calculation unit 132 may calculate the predicted value of a life satisfaction level by country using the growth curve based on a logistic curve or a Gompertz curve. Then, the output unit 133 outputs the predicted value of a life satisfaction level by country (process S103), and terminates the step.

### Second Embodiment

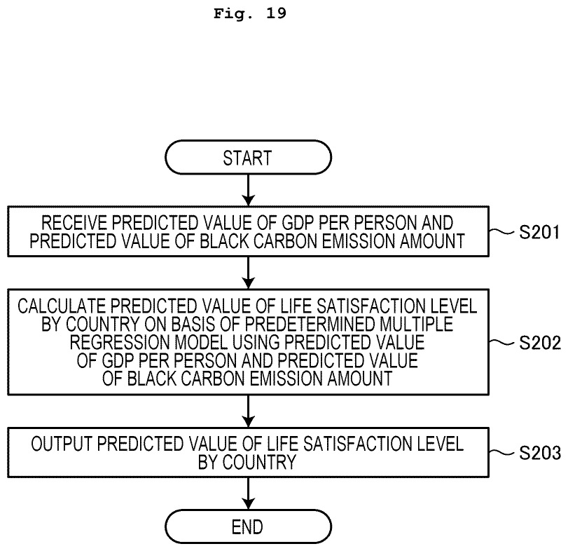

Next, the processing procedure of the second embodiment will be described with reference to FIG. 19. FIG. 19 is a diagram illustrating an example of the flowchart of the prediction method according to the second embodiment.

The reception unit 131 receives the predicted value of GDP per capita and the predicted value of a black carbon emission amount (process S201). The calculation unit 132 calculates the predicted value of a life satisfaction level by country on the basis of the predetermined multiple regression model using the predicted value of GDP per capita and the predicted value of a black carbon emission amount (process S202). Then, the output unit 133 outputs the predicted value of a life satisfaction level by country (process S203), and terminates the process.

### Third Embodiment

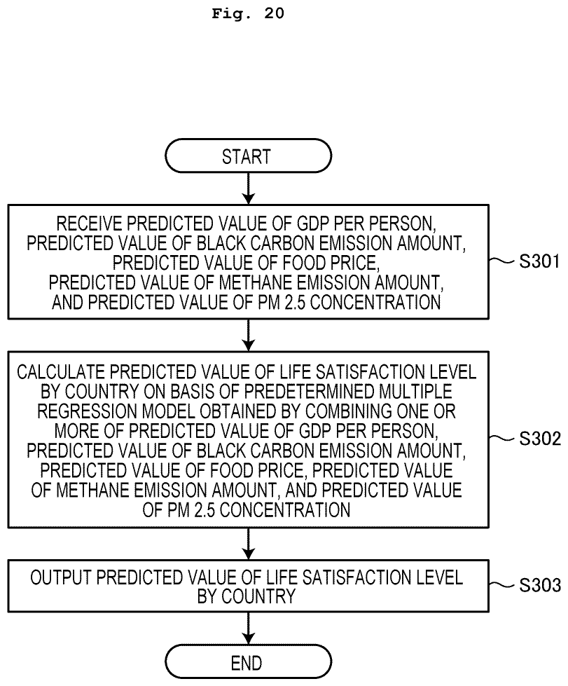

Next, the processing procedure of the third embodiment will be described with reference to FIG. 20. FIG. 20 is a diagram illustrating an example of the flowchart of the prediction method according to the third embodiment.

The reception unit 131 receives the predicted value of GDP per capita, the predicted value of a black carbon emission amount, the predicted value of a food price, the predicted value of a methane emission amount, and the predicted value of PM2.5 concentration (process S301).

The calculation unit 132 calculates the predicted value of a life satisfaction level by country on the basis of the predetermined multiple regression model including the predicted value of GDP per capita, and one of or a combination of a plurality of the predicted value of a black carbon emission amount, the predicted value of a food price, the predicted value of a methane emission amount, and the predicted value of PM2.5 concentration (process S302).

The output unit 133 outputs the predicted value of a life satisfaction level by country (process S303), and terminates the process.

### Fifth Embodiment

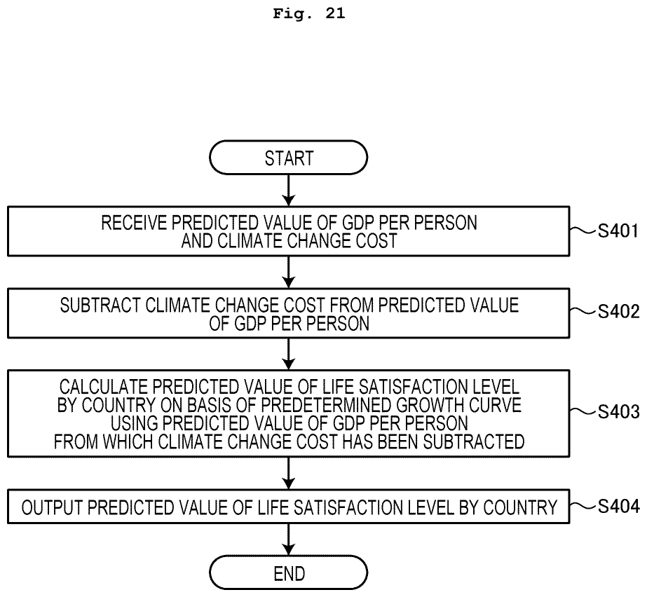

Next, the processing procedure of the fifth embodiment will be described with reference to FIG. 21. FIG. 21 is a diagram illustrating an example of the flowchart of the prediction method according to the fifth embodiment.

The reception unit 131 receives the predicted value of GDP per capita and the climate change cost (process S401). The calculation unit 132 subtracts the climate change cost from the predicted value of GDP per capita (process S402).

The calculation unit 132 calculates the predicted value of a life satisfaction level by country on the basis of the predetermined growth curve, using the predicted value of GDP per capita from which the climate change cost has been subtracted (process S403). Then, the output unit 133 outputs the predicted value of a life satisfaction level by country (process S404), and terminates the process,

### Sixth Embodiment

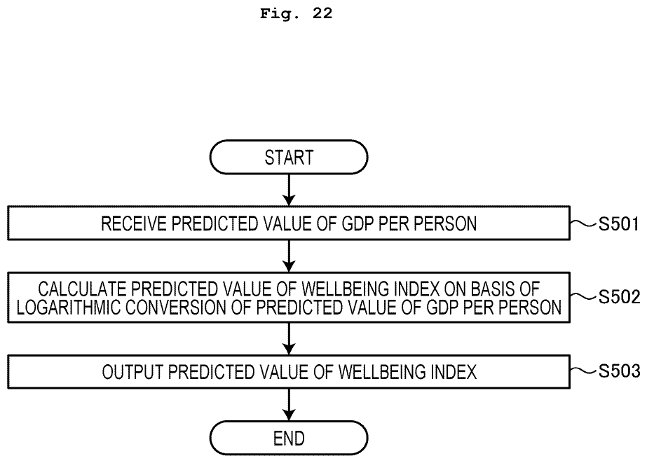

Next, the processing procedure of the sixth embodiment will be described with reference to FIG. 22. FIG. 22 is a diagram illustrating an example of the flowchart of the prediction method based on logarithmic conversion according to the sixth embodiment.

The reception unit 131 receives the predicted value of GDP per capita (process S501). The calculation unit 132 calculates the predicted value of the wellbeing index on the basis of logarithmic conversion of the predicted value of GDP per capita (process S502). Then, the output unit 133 outputs the predicted value of the wellbeing index (process S503), and terminates the process.

### Seventh Embodiment

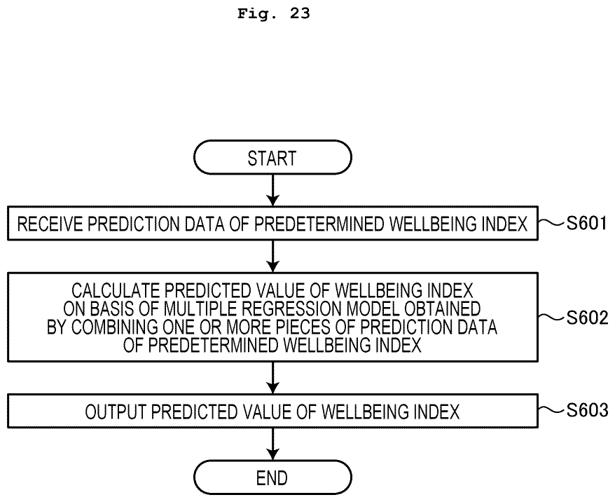

Next, the processing procedure of the seventh embodiment will be described with reference to FIG. 23. FIG. 23 is a diagram illustrating an example of the flowchart of the prediction method based on the multiple regression model according to the seventh embodiment.

The reception unit 131 receives the prediction data of a predetermined wellbeing index (process S601), The calculation unit 132 calculates the predicted value of the wellbeing index on the basis of the multiple regression model including one piece of or a combination of a plurality of pieces of the prediction data of the predetermined wellbeing index (process S602). Then, the output unit 133 outputs the predicted value of the wellbeing index (process S603), and terminates the process.

### [10. Effects]

Hereinafter, effects provided by the prediction device 100 according to the present embodiment will be described.

Conventionally, in order to scientifically predict and evaluate an influence of global warming on water circulation, food production, and natural disasters on society and economy, simulation based on a comprehensive evaluation model integrating various knowledge of weather, human activities, and the like has been conducted. An object predicted by a conventional integrated evaluation model is an objective index regarding society and economy, such as water consumption, food prices, food production amounts, and energy prices. Meanwhile, there is a part in which human affluence cannot be measured only by an objective economic index, and in the World Happiness Report, the degree of happiness is investigated and reported on a global scale, using subjective evaluation results based on questionnaire surveys as important factors.

It is desirable that social measures and policies also consider the degree of happiness (life satisfaction level) as an important index, and many studies have been conducted on factors that affect the subjective degree of happiness. Note that, in recent years, it is considered that there are not only factors in which a clear correlation is recognized but also factors that potentially affect. However, in conventional studies, studies of prediction of a future subjective degree of happiness have not been conducted in evaluation of a relationship between a social indicators value actually observed at present or in the past and the subjective degree of happiness. Therefore, there is a problem that it is difficult to utilize the subjective degree of happiness as an index for studying measures and policies.

In summary, in the conventional technology and study, the predictable index is limited to the objective index regarding society and economy, and the future prediction of the subjective degree of happiness is not performed. Further, what has been studied in the conventional degree of happiness study is limited to the past and the present subjective degree of happiness, and the future prediction of the subjective degree of happiness has not been performed. Further, in the conventional degree of happiness study, the life satisfaction level can be predicted by a logarithmic value of an individual's income (for example, GDP per capita or the like), but the accuracy is not sufficient, and there is room for improvement.

Further, in the integrated evaluation model that comprehensively evaluates weather, socio-economic activities, and the like, an objective index is modeled and future prediction is performed by simulation, but the subjective degree of happiness, which is familiar and important for an individual's life, is not sufficiently considered.

Therefore, the reception unit 131 of the prediction device 100 according to the present embodiment receives the information regarding predetermined future prediction.

Then, the calculation unit 132 of the prediction device 100 calculates the predicted value of a life satisfaction level by country, using the information regarding predetermined future prediction.

Specifically, the reception unit 131 of the prediction device 100 receives the predicted value of GDP per capita as the information regarding predetermined future prediction. Then, the calculation unit 132 of the prediction device 100 calculates the predicted value of a life satisfaction level by country, using the growth curve based on a logistic curve or a Gompertz curve, using the predicted value of GDP per capita.

Further, the reception unit 131 of the prediction device 100 receives the predicted value of GDP per capita and the predicted value of a black carbon emission amount, as the information regarding predetermined future prediction. Then, the calculation unit 132 of the prediction device 100 calculates the predicted value of a life satisfaction level by country on the basis of the predetermined multiple regression model obtained by adding the predicted value of GDP per capita and the predicted value of a black carbon emission amount.

Further, the reception unit 131 of the prediction device 100 receives the predicted value of GDP per capita, the predicted value of a black carbon emission amount, the predicted value of a food price, the predicted value of a methane emission amount, and the predicted value of PM2.5 concentration, as the information regarding predetermined future prediction. Then, the calculation unit 132 of the prediction device 100 calculates the predicted value of a life satisfaction level by country on the basis of the predetermined multiple regression model including the predicted value of GDP per capita, and one of or a combination of a plurality of the predicted value of a black carbon emission amount, the predicted value of a food price, the predicted value of a methane emission amount, and the predicted value of PM2.5 concentration,

Further, the prediction device 100 receives the information regarding predetermined future prediction predicted for each of a plurality of socio-economic scenarios,

Therefore, according to the present embodiment, the prediction device 100 has an effect of enabling utilization of the index of the subjective degree of happiness as an index for studying measures and policies.

As a result, it becomes possible to perform the future prediction of a subjective life satisfaction level (degree of happiness) on the basis of the objective index that can be predicted for each socio-economic scenario, and it becomes possible to utilize the subjective life satisfaction level as an index for studying formulation of measures and policies.

For example, the prediction device 100 of the present embodiment predicts the life satisfaction level, which is one index of the subjective degree of happiness, on the basis of a linear multiple regression model based on the GDP per capita and the black carbon emission amount. The logarithmic conversion is used in the prior study, whereas in the present embodiment, the GDP per capita is converted by the predetermined growth curve, whereby an effect of improving the accuracy of the future prediction of the life satisfaction level is achieved.

Meanwhile, in the model created this time, the black carbon emission amount has a negative influence on the life satisfaction level, but the most strongly influenced and dominant is the GDP per capita, and the direction is different from that of a sustainable society. In this way, the prediction device 100 provides an effect of enabling presentation of the degree of happiness index other than the life satisfaction level, which leads to a sustainable society,

Further, the reception unit 131 receives the predicted value of GDP per capita and the climate change cost as the information regarding predetermined future prediction (prediction data). The calculation unit 132 subtracts the climate change cost from the predicted value of GDP per capita. Then, the calculation unit 132 calculates the predicted value of a life satisfaction level by country, using the growth curve based on a logistic curve or a Gompertz curve, using the predicted value of GDP per capita from which the climate change cost has been subtracted,

Therefore, the prediction device 100 can predict the predicted value in which the influence of climate change has been taken into consideration. As a result, the prediction device 100 can predict the predicted value of the life satisfaction level by country with higher accuracy than the predicted value of the life satisfaction level by Country predicted in the first embodiment.

Further, the calculation unit 132 calculates the predicted value of the wellbeing index as the subjective degree of happiness using the information regarding predetermined future prediction (prediction data) received by the reception unit 131.

For example, the calculation unit 132 calculates the predicted value of the wellbeing index on the basis of the logarithmic conversion of the predicted value of GDP per capita received by the reception unit 131. Therefore, the prediction device 100 accurately predicts the predicted value of the wellbeing index by using the input predicted value of GDP per capita, thereby achieving an effect of enabling utilization of the wellbeing index as an index for studying measures and policies.

Furthermore, for example, the calculation unit 132 calculates the predicted value of the wellbeing index on the basis of the multiple regression model including one piece of or a combination of a plurality of pieces of the information regarding predetermined future prediction (prediction data) received by the reception unit 131. Specifically, the calculation unit 132 calculates the predicted value of the wellbeing index on the basis of the predetermined multiple regression model including the predicted value of GDP per capita, and one of or a combination of a plurality of the predicted value of water stress, the predicted value of water safety, the predicted value of air quality, the predicted value of food supply, the predicted value of an income inequality, and the predicted value of an area ratio between a rural area and an urban area.

Therefore, the prediction device 100 calculates the predicted value on the basis of the multiple regression model obtained by combining a plurality of pieces of input prediction data, thereby enabling prediction with higher accuracy than the method based on logarithmic conversion of single piece of data (predicted value of GDP per capita).

### <Modification>

Hereinafter, a modification implemented by the prediction device 100 according to the present embodiment will be described.

### (Data, etc.)

The socio-economic scenarios, GDP performance data per person by country, social indicators performance data, climate record data, predicted value of GDP per capita, predicted value of a social indicators, predicted value of climate indicators, subjective degree of happiness, predicted value of a life satisfaction level by country, predicted value of a wellbeing index, predicted value of food price, predicted value of a methane emission amount, predicted value of a black carbon emission amount, predicted value of a methane emission amount, predicted value of PM2.5 concentration, cost generated by climate change, predicted value of water stress, predicted value of water safety, predicted value of air quality, predicted value of food supply (grain), predicted value of an income inequality, predicted value of an area ratio between a rural area and an urban area, questionnaire result of self-evaluation regarding health, suicide rate, DALY that is an index representing a comprehensive health state, names of the functional units of the prediction device 100, steps, processes, names of the steps and processes, and the like used in the description of the embodiments are merely examples, and can be arbitrarily changed.

### (Flowchart, etc.)

Each step in the flowchart or the like may be replaced and performed within a range without inconsistency, or there may be a step that is not performed. Furthermore, the conjunctions such as “next”, “continue”, “further”, “at this time”, “in this case”, and the like in the description of the flowchart do not limit the execution order and timing of the processing in the flowchart,

### (System)

The processing procedure, the control procedure, the specific name, and the information including various kinds of data and parameters illustrated in the document and the drawings can be arbitrarily changed unless otherwise specified.

In addition, each component of each device that has been illustrated is functionally conceptual, and is not necessarily physically configured as illustrated. That is, specific forms of distribution and integration of the devices are not limited to those illustrated in the drawings. That is, all or a part thereof can be functionally or physically distributed and integrated in any unit according to various loads, usage conditions, and the like. For example, learning processing and classification processing may be implemented by different devices, and both may be connected via a communication unit.

### [11. Hardware Configuration]

Each component of each device illustrated in the drawings is functionally conceptual and does not necessarily need to be physically configured as illustrated. “That is, a specific form of distribution and integration of each device is not limited to the illustrated form, and all or a part thereof can be functionally or physically distributed and integrated in any unit according to various loads, usage conditions, and the like. Furthermore, all or any part of each processing function performed in each device can be implemented by a CPU and a program analyzed and executed by the CPU, or can be implemented as hardware by a wired logic.

Moreover, among the pieces of processing described in the present embodiment, all or a part of the processing described as being automatically performed can be manually performed by a known method. The processing procedures, control procedures, specific names, and information including various types of data and parameters described in the drawings can be freely changed unless otherwise specified.

[Program] As an embodiment, various devices included in the prediction device 100 can be implemented by installing the above-described prediction program as packaged software or online software in a desired computer. For example, by causing the information processing device to execute the above-described prediction program, it is possible to cause the information processing device to function as various devices constituting the prediction device 100. The information processing device described here includes a desktop personal computer or a laptop personal computer. In addition, the information processing device also includes a mobile communication terminal such as a smartphone or a mobile phone, a slate terminal such as a personal digital assistant (PDA), and the like.

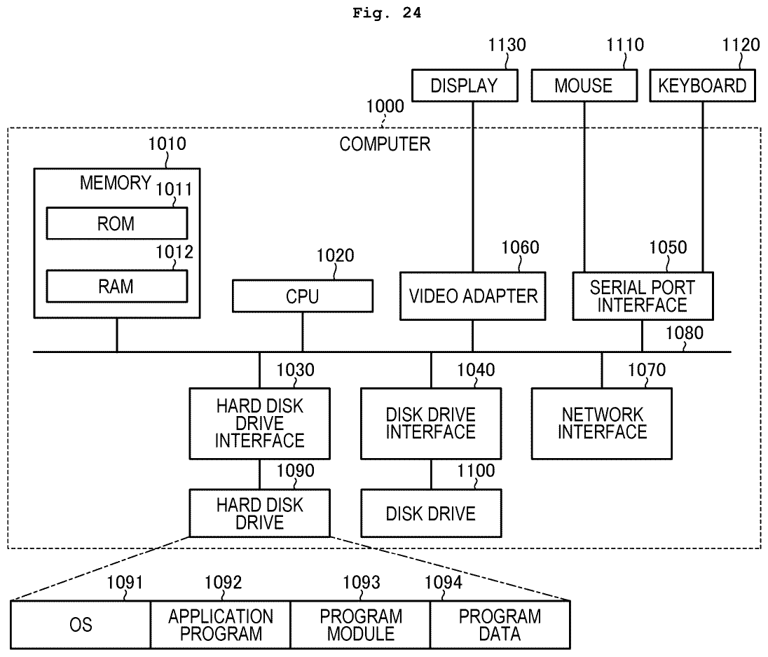

FIG. 24 is a diagram illustrating an example of a computer on which the prediction device 100 according to the present embodiment is implemented. A computer 1000 includes, for example, a memory 1010 and a CPU 1020. The computer 1000 also includes a hard disk drive interface 1030, a disk drive interface 1040, a serial port interface 1050, a video adapter 1060, and a network interface 1070. These units are connected to each other by a bus 1080.

The memory 1010 includes a read only memory (ROM) 1011 and a RAM 1012. The ROM 1011 stores, for example, a boot program such as a basic input output system (BIOS). The hard disk drive interface 1030 is connected with a hard disk drive 1090. The disk drive interface 1040 is connected with a disk drive 1100. For example, a removable storage medium such as a magnetic disk or an optical disk is inserted into the disk drive 1100. The serial port interface 1050 is connected with, for example, a mouse 1110 and a keyboard 1120. The video adapter 1060 is connected with, for example, a display 1130.

The hard disk drive 1090 stores, for example, an OS 1091, an application program 1092, a program module 1093, and program data 1094. That is, a program that defines each piece of processing of the various devices included in the prediction device 100 is implemented as the program module 1093 in which codes executable by a computer are described. The program module 1093 is stored in, for example, the hard disk drive 1090. For example, the program module 1093 for executing processing similar to the processing of the functional configuration of the various devices included in the prediction device 100 is stored in the hard disk drive 1090. Note that the hard disk drive 1090 may be replaced with a solid state drive (SSD).

In addition, setting data used in the processing in the embodiments described above is stored in, for example, the memory 1010 or the hard disk drive 1090 as the program data 1094. Then, the CPU 1020 reads the program module 1093 and the program data 1094 stored in the memory 1010 or the hard disk drive 1090 to the RAM 1012 as necessary and executes the processing in the embodiments described above.

Note that the program module 1093 and the program data 1094 are not limited to being stored in the hard disk drive 1090, and may be stored in, for example, a removable storage medium and read by the CPU1020 via the disk drive 1100 or the like. Alternatively, the program module 1093 and the program data 1094 may be stored in another computer connected via a network (LAN, wide area network (WAN), or the like). Then, the program module 1093 and the program data 1094 may be read by the CPU 1020 from another computer via the network interface 1070.

### [12. Others]

Although the present embodiment has been described above, the present embodiment is not limited by the description and drawings constituting a part of the disclosure. That is, other embodiments, examples, operational technologies, and the like made by those skilled in the art or the like on the basis of the present embodiment are all included in the scope of the present embodiment.

### REFERENCE SIGNS LIST

10 Predicted value of GDP per capita
11 Predicted value of a social indicators
12 Predicted value of a climate index
13 Predicted value of a life satisfaction level by country
20 Socio-economic scenario
21 GDP per capita by country performance data
22 Social indicators performance data
23 Climate record data
30 List
100 Prediction device
110 Communication unit
120 Storage unit
121 Economic index storage unit
122 Life satisfaction level predicted value storage unit
123 Social indicators storage unit
124 Climate index storage unit
125 Prediction data storage unit
126 Wellbeing index predicted value storage unit
130 Control unit
131 Reception unit
132 Calculation unit
133 Output unit
1000 Computer
1010 Memory
1011 ROM
1012 RAM
1020 CPU
1030 Hard disk drive interface
1040 Disk drive interface
1050 Serial port interface
1060 Video adapter
1070 Network interface
1080 Bus
1090 Hard disk drive
1091 OS
1092 Application program
1093 Program module
1094 Program data
1100 Disk drive
1110 Mouse
1120 Keyboard

1. A prediction device comprising:
a memory; and
processing circuitry configured to:
receive information regarding predetermined future prediction; and
calculate a predicted value of a life satisfaction level by country, using the information regarding predetermined future prediction.

2. The prediction device according to claim 1, wherein the processing circuitry is further configured to:
receive a predicted value of GDP per capita as the information regarding predetermined future prediction, and
calculate the predicted value of a life satisfaction level by country, using a growth curve based on a logistic curve or a Gompertz curve, using the predicted value of GDP per capita.

3. The prediction device according to claim 1, wherein the processing circuitry is further configured to:
receive a predicted value of GDP per capita and a predicted value of a black carbon emission amount as the information regarding predetermined future prediction, and
calculate the predicted value of a life satisfaction level by country on a basis of a predetermined multiple regression model obtained by adding the predicted value of GDP per capita and the predicted value of a black carbon emission amount.

4. The prediction device according to claim 1, wherein the processing circuitry is further configured to:
receive a predicted value of GDP per capita, a predicted value of a black carbon emission amount, a predicted value of a food price, a predicted value of a methane emission amount, and a predicted value of PM2.5 concentration as the information regarding predetermined future prediction, and
calculate the predicted value of a life satisfaction level by country on a basis of a predetermined multiple regression model including the predicted value of GDP per capita, and one of or a combination of a plurality of the predicted value of a black carbon emission amount, the predicted value of a food price, the predicted value of a methane emission amount, and the predicted value of PM2.5 concentration.

5. The prediction device according to claim 1, wherein the processing circuitry is further configured to receive the information regarding predetermined future prediction predicted for each of a plurality of socio-economic scenarios.

6. A prediction method executed in a prediction device, the prediction method comprising:
receiving information regarding predetermined future prediction; and
calculating a predicted value of a life satisfaction level by country, using the information regarding predetermined future prediction.

7. A non-transitory computer-readable recording medium storing therein a prediction program that causes a computer to execute a process comprising:
receiving information regarding predetermined future prediction; and
calculating a predicted value of a life satisfaction level by country, using the information regarding predetermined future prediction.

8. The prediction device according to claim 1, wherein the processing circuitry is further configured to:
receive a predicted value of GDP per capita and a cost generated by climate change as the information regarding predetermined future prediction, and
subtract the cost generated by climate change from the predicted value of GDP per capita, and calculate the predicted value of a life satisfaction level by country, using a growth curve based on a logistic curve or a Gompertz curve, using the predicted value of GDP per capita from which the cost generated by climate change has been subtracted.

9-16. (canceled)
